(19) United States
(12) Patent Application Publication
(10) Pub. No.: US 2010/0279122 A1
(43) Pub. Date: Nov. 4, 2010

(54) Benzimidazole, Benzoxazole and Benzothiazole Derivatives, Optical Film Comprising them and Method of Producing thereof

(75) Inventors: Alexey Nokel, Moscow (RU); Pavel I. Lazarev, London (GB)

Correspondence Address: HOUST CONSULTING (Kont), P.O. BOX 2688, SARATOGA, CA, 95070-0688, US

(73) Assignee: CRYSOPTIX KK, Tokyo (JP)

(21) Appl. No.: 12/377,095

(22) PCT Filed: Aug. 16, 2007
(86) PCT No.: PCT/GB07/03120

§ 371 Date: Feb. 10, 2009

(30) Foreign Application Priority Data
Aug. 16, 2006 (GB) 0616359.6
May 18, 2007 (GB) 0709605.0

(51) Int. Cl. C07D 403/14; C07D 235/20; B05D 5/06; B32B 17/06; B32B 27/06

(52) U.S. Cl. 428/426; 544/198; 548/305.4; 427/162; 428/704

(57) ABSTRACT

The present invention is relates to the synthesis of predominantly planar heterocyclic organic compound and the manufacture of optical films based on these compounds. Said organic compound has the general structural formula where Het is a predominantly planar heterocyclic molecular system possessing hydrophilic properties; B is a binding group; p is the number in the range from 3 to 8; S is a group providing solubility of the organic compound; m is a number in the range from 0 to 8. Said organic compound is transparent for electromagnetic radiation in the visible spectral range from 400 to 700 nm, and a solution of the compound or a salt thereof is capable of forming a substantially transparent optical layer on a substrate, with the heterocyclic molecular planes oriented predominantly parallel to the substrate surface.

The present invention relates generally to the field of organic chemistry and particularly to organic films with phase-retarding properties for displays.

In connection with polarization, compensation and retardation layers, films, or plates described in the present application, the following definitions of terms are used throughout the text.

The term optical axis refers to a direction in which propagating light does not exhibit birefringence.

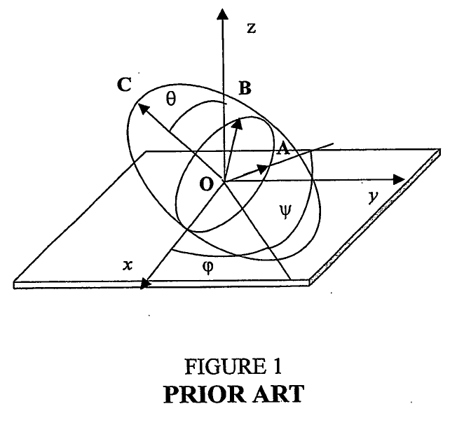

Any optically anisotropic medium is characterized by its second-rank dielectric permittivity tensor. The classification of compensator plates is tightly connected to orientations of the principal axes of a particular permittivity tensor with respect to the natural coordinate frame of the plate. The natural xyz coordinate frame of the plate is chosen so that the z-axis is parallel to the normal direction and the xy plane coincides with the plate surface. FIG. 1 demonstrates a general case when the principal axes (A, B, C) of the permittivity tensor are arbitrarily oriented relative to the xyz frame.

Orientations of the principal axes can be characterized using three Euler's angles (θ, φ, ψ) which, together with the principal permittivity tensor components (ε_{A}, ε_{B}, ε_{C}), uniquely define different types of optical compensators (FIG. 1). The case when all the principal components of the permittivity tensor have different values corresponds to a biaxial compensator, whereby the plate has two optical axes. For instance, in the case of ε_{A}<ε_{B}<ε_{C}, these optical axes are in the plane of C and A axes on both sides from the C axis. In the uniaxial limit, when ε_{A}=ε_{B}, we have a degenerate case when the two axes coincide and the C axis is a single optical axis.

The zenith angle θ between the C axis and the z axis is most important in the definitions of various compensator types. There are several important types of compensator plates, which are most frequently used in practice.

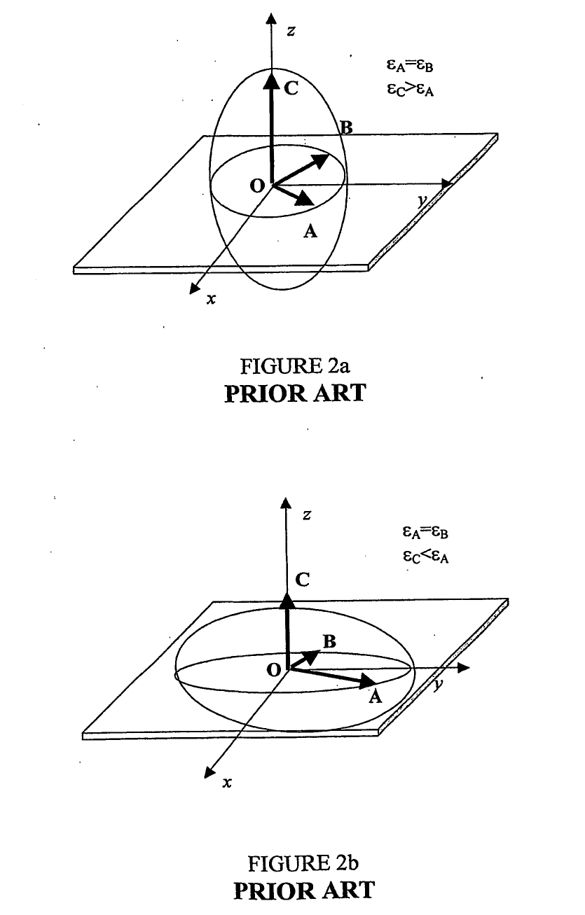

A uniaxial C-plate is defined by the Euler angle θ=0 and ε_{A}=ε_{B}≠ε_{c}. In this case, the principal C axis (extraordinary axis) is normal to the plate surface (xy plane). In cases of ε_{A}=ε_{B}<ε_{C}, the plate is called “positive C-plate”. On the contrary, if ε_{A}=ε_{B}>ε_{C}, the plate is referred to as the “negative C-plate”. FIG. 2 shows the orientation of the principal axes of a particular permittivity tensor with respect to the natural coordinate frame of the positive (a) and negative (b) C-plate. The axes OA and OB located in a xy plane are equivalent. Therefore conditions between refractive indices na and nb remain fair at replacement of an index na by an index nb and at replacement of an index nb by an index na.

Generally when the permittivity tensor components (ε_{A}, ε_{B}, and ε_{C}) are complex values, the principal permittivity tensor components (ε_{A}, ε_{B}, and ε_{C}), the refraction indices (na, nb, and nc), and the absorption coefficients (ka, kb, and kc) meet the following conditions: na=Re[(ε_{A})^{1/2}], nb=Re[(ε_{B})^{1/2}], nc=Re[(ε_{C})^{1/2}], ka=lm[(ε_{A})^{1/2}], kb=lm[(ε_{B})^{1/2}], kc=lm[(ε_{C})^{1/2}].

Liquid crystals are widely used in electronic displays. In such display systems, a liquid crystal cell is typically situated between a pair of polarizer and analyzer plates. The incident light is polarized by the polarizer and transmitted through a liquid crystal cell, where it is affected by the molecular orientation of the liquid crystal that can be controlled by applying a bias voltage across the cell. Then, the altered light is transmitted through the analyzer. By employing this scheme, the transmission of light from any external source, including ambient light, can be controlled. The energy required to provide for this control is generally much lower than that required for controlling the emission from luminescent materials used in other display types such as cathode ray tubes (CRTs). Accordingly, liquid crystal technology is used in a number of electronic imaging devices, including (but not limited to) digital watches, calculators, portable computers, and electronic games, for which small weight, low power consumption, and long working life are important.

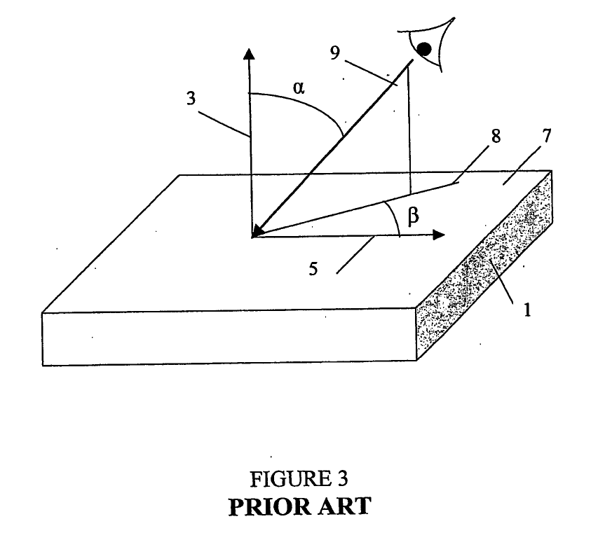

The contrast, color reproduction, and stable gray scale intensities are important quality characteristics of electronic displays, which employ liquid crystal technology. The primary factor determining the contrast of a liquid crystal display (LCD) is the propensity for light to “leak” through liquid crystal elements or cells, which are in the dark or “black” pixel state. In addition, the optical leakage and, hence, the contrast of an LCD also depend on the direction from which the display screen is viewed. Typically, the optimum contrast is observed only within a narrow viewing angle range centered about the normal (α=0) to the display and falls off rapidly as the polar viewing angle α is increased. FIG. 3 illustrates the definition of the viewing angle direction. In color displays, the leakage problem not only degrades the contrast but also causes color or hue shifts with the resulting degradation of color reproduction.

LCDs are replacing CRTs as monitors for television (TV) sets, computers (such as, for example, notebook computers or desktop computers), central control units, and various devices, for example, gambling machines, electro-optical displays, (such as displays of watches, pocket calculators, electronic pocket games), portable data banks (such as personal digital assistants or of mobile telephones). It is also expected that the number of LCD television monitors with a larger screen size will sharply increase in the near future. However, unless problems related to the effect of viewing angle on the coloration, degradation in, contrast, and the inversion of brightness are solved, the replacement of traditional CRTs by LCDs will be limited.

In the normally white display configuration, a 90°-twist nematic cell is placed between crossed polarizers, so that the transmission axis of each polarizer is parallel to the orientation of the director of liquid crystal molecules in the region of the cell adjacent to it. This reverses the sense of bright and dark areas as compared to that in the normally black display. The unenergized (unbiased) areas appear bright in a normally white display, while the energized areas appear dark. The problem of ostensibly dark areas appearing light when viewed at large angles still occurs. But the reason for this is different and its correction requires a different type of the optical compensating element. In the energized areas, the liquid crystal molecules tend to align in the direction of an applied electric field. If this alignment were perfect, all the liquid crystal molecules in the cell would have their long axes normal to the substrate glass plate. This arrangement, known as the homeotropic configuration, exhibits the optical symmetry of a positively birefringent C-plate. In the energized state, the normally white display appears isotropic to normally incident light, which is blocked by the crossed polarizers.

The loss of contrast with increasing viewing angle occurs because the homeotropic liquid crystal layer does not appear isotropic to light propagating at an angle relative to the normal direction. Light directed at a nonzero angle relative to the normal propagates in two modes due to the birefringence of the layer, with a phase delay between these modes that increases with the light incidence angle. This phase dependence on the incidence angle introduces an ellipticity into the polarization state, which is then incompletely extinguished by the second polarizer, giving rise to light leakage. Because of the C-plate symmetry, the birefringence has no azimuthal dependence. Obviously, what is needed is an optical compensating element, also with a C-plate symmetry, but with a negative birefringence. Such a compensator would introduce a phase delay opposite in sign to that caused by the liquid crystal layer, thereby restoring the original polarization state and allowing the light to be blocked by the output polarizer.

No methods were available for stretching or compressing polymers so, as to obtain the films of large area with negative C-plate optical symmetry and the required degree of uniformity; nor was it possible to form a compensator from a negatively birefringent crystal such as sapphire. In order for such a compensator to be effective, the phase retardation of such a plate must be of the same magnitude as that of the liquid crystal and it would also have to change with viewing angle at the same rate as does the phase retardation in the liquid crystal. These constraints imply that the thickness of the negative C-plate would be on the order of 10 μm, making such an approach very difficult to implement because it would require polishing of an extremely thin plate having the correct (negative) birefringence while ensuring that the surfaces of the plate remain parallel. Since such displays are relatively large in size, the availability of a negatively birefringent crystal of sufficient size would also be a major difficulty.

There is one known C-plate, which consist of alternating thin films of materials with different indices of refraction. Such a layered structure can operate as an artificial birefringent thin plate. A multilayer compensator fabricated in this manner can be made to exhibit negative birefringence; moreover, the desired birefringence of the multilayer structure can be tailored precisely by choosing proper layer thicknesses and materials. The main drawback of said C-plate is a high cost of its production.

Uncompensated full colors LCDs typically exhibit a large variation in chromaticity over the field of view. Consequently, an area that appears one color when viewed at normal incidence may appear less saturated or may even appear as its complementary color when viewed at large angles. These results from the same physical mechanism which causes diminished contrast at large angles, that is, unwanted light leakage through the ostensibly dark areas.

The present invention provides a practical solution to the need for such a compensator. The idea is to create crystalline retarder films with high optical parameters on the basis of organic compounds. The creation of the crystalline retarders of such a kind requires a special arrangement of molecules in the multilayer film. Organic molecules have to be parallel to the substrate surface.

There is a known organic quasi-epitaxial method intended for the formation of optoelectronic devices (see. U.S. Pat. No. 5,315,129, Forrest et al., Organic Optoelectronic Devices and Methods). According to this method, the planes of organic molecules are oriented parallel to the substrate surface. A quasi-epitaxial optoelectronic device structure comprises a substrate, the first layer deposited onto said substrate, and the second layer deposited onto the first layer. Said first layer represents a planar crystalline film of an organic aromatic semiconductor compound, which is selected from a list of organic compounds comprising polyacenes, porphyrins, and their derivatives. Said second layer also represents a planar crystalline film of an organic aromatic semiconductor, whose chemical composition (generally, different from that of the first layer) is also selected from a list of organic compounds comprising polyacenes, porphyrins, and their derivatives. The first and second layers have crystalline structures, which are in a certain relationship with each other. In particular, the first and second, layers can be independently selected from a list comprising 3,4,9,10-perylenetetracarboxylic dianhydride (PTCDA), 3,4,7,8-naphthalenetetracarboxylic dianhydride (NTCDA), copper phthalocyanine, 3,4,9,10-perylenetetracarboxylic acid bis-benzimidazole, and -oxadiazole derivatives. Organic optoelectronic devices have been grown using the organic molecular beam deposition technology. The organic substances have been deposited as ultrathin layers only 10 Angstroms (Å) thick using organic molecular beam deposition methods. PTCDA and NTCDA have been identified as excellent materials for the manufacture of organic optoelectronic IC devices, but any planar organic aromatic semiconductor capable of readily forming a crystalline structure may be used. The preferred method of the prior art employs a chamber, containing an inorganic substrate made of an appropriate material for making electrical contact to the organic structures, and sources of PTCDA and NTCDA. The pressure in the chamber is maintained on a level generally below 10⁻⁶ Torr. The substrate is spaced from the source of component materials by a minimum distance of 10 cm. During deposition, the substrate is kept at a temperature below 150K, while the PTCDA and NTCDA sources are alternatively heated.

Despite all the advantages of said quasi-epitaxial growth method (see U.S. Pat. Nos. 6,451,415 and 5,315,129), it is not free of drawbacks. According to said known method, a constant temperature regime and vacuum level have to be maintained in the chamber throughout the epitaxial growth process. Any breakdowns in the temperature and vacuum regime lead to the appearance of defects in the growing layer, whereby both crystallographic parameters and the orientation of molecular layer exhibit changes. This sensitivity of the process with respect to instability of the technological parameters can be also considered as a disadvantage of said known method, which is especially significant in the case of deposition of relatively thick (1 to 10 μm) epitaxial layers.

Another disadvantage of said method is the need for sophisticated technological equipment. The reactor chamber must hold an ultrahigh vacuum (down to 10⁻⁶-10⁻¹⁰ Torr) and must withstand considerable temperature gradients between closely spaced zones. The equipment must include the means of heating sources and cooling substrates, a complicated pumping stage, and facilities for gas admission, temperature and pressure monitoring, and technological process control. The high vacuum requirements make the process expensive and limit the substrate dimensions.

One more disadvantage of said known technology is limitation on the choice of substrate materials: only substances retaining their physical, mechanical, optical, and other properties under the conditions of large pressure differences, high vacuum, and considerable temperature gradients can be employed.

The production of a two-dimensional bimolecular surface structure using weak noncovalent interactions has been demonstrated and the products were characterized by scanning tunneling microscopy (see L. Scudiero et al., “A Self-Organized Two-Dimensional Bimolecular Structure”, J. Phys. Chem. B, 107, 2903-2909 (2003)). This work follows closely the ideas of three-dimensional crystal engineering and applies the concepts of supramolecular reactants (synthons) to molecular systems constrained to two dimensions by physical adsorption (physisdrption) on a conducting surface. A well-ordered planar structure that self-assembles due to the fluorine-phenyl interactions has been demonstrated. This study provides an example of the systematic design of self-organized layers. Fully fluorinated cobalt phthalocyanine (F16CoPc) films thermally deposited onto gold were characterized by reflection-absorption infrared spectroscopy (RAIRS), X-ray and ultraviolet photoelectron spectroscopy (XPS and UPS), and scanning tunneling microscopy (STM). The UPS spectra of thin films of CoPc, F16CoPc, and nickel tetraphenylporphyrin (NiTPP) on gold were measured and their relative surface charges were compared. STM images of single molecular layers of F16CoPc, NiTPP, and NiTPP-F16CoPc and NiTPP-CoPc mixtures were obtained. It was found that, while NiTPP-F16CoPc spontaneously formed a well-ordered 1:1 structure, NiTPP-CoPc formed a two-dimensional solid solution.

Ultrathin films prepared from certain inorganic and organic materials have drawn increasing interest as hybrid nanocomposite materials. The formation of nanostructured ultrathin films of montmorillonite clay (MONT) and a bicationic sexithiophene derivative (6TN) was investigated using the layer-by-layer self-assembly approach (see X. Fan, J. Locklin, J. Ho Youk, et al., Nanostructured Sexithiophene/Clay Hybrid Mutilayers: A Comparative Structural and Morphological Characterization, Chem. Mater., 14, 2184-2191 (2002)). The main goal was to investigate the structure and layer ordering in these films suitable for future applications in organic semiconductor devices. The structure and morphology of 6TNIMONT multilayer films prepared from pure water and 0.1 M NaCl systems have been compared. The 6TN amphiphile showed unique aggregation behaviour both in solution and on the surface, which changed in the presence of salts and THF as a cosolvent. On clay surfaces, the 6TN aggregates deposited from saline solutions exhibited a more uniform size distribution and surface coverage as compared to those obtained from a pure water system. This was verified by UV-VIS spectra, X-ray diffraction (XRD), and atomic force microscopy (AFM). The idea of incorporating more 6TN species adsorbed on the surface so as to obtain a smoother surface morphology can be of great significance in semiconductor device fabrication.

The available literature offers no examples of films with the vertical orientation of stacks prepared by a low-cost and effective way of solution application onto the substrate. Using lyotropic liquid crystal (LLC) solutions of sulfoderivatives, we usually obtain films with the horizontal orientation of stacks (see: U.S. Pat. Nos. 5,739,296 and 6,049,428 and the following publications: P. Lazarev et al., X-ray Diffraction by Large Area Organic Crystalline Nanofilms, Molecular Materials, 14(4), 303-311 (2001), and Y. Bobrov, Spectral Properties of Thin Crystal Film Polarizers, Molecular Materials, 14(3), 191-203 (2001)).

On the other hand, it is known from the literature that some molecules are capable of forming regularly arranged planar fragments (supramolecules) on a substrate surface, being deposited from solutions in water and various organic solvents, and that hydrogen bonding (H-bonding) is the driving force for the formation of such planar supramolecules. This phenomenon was observed for heterocyclic amines, amides, and carboxylic acids. The type of the obtained monolayer structure depends on the molecular structure, the solvent, and the surface activity. The layer structures of various types—stable and unstable, dense and loose—can be obtained using different molecular structures and conditions.

There are many novel adsorbate-pmd substrate systems, which are known to exhibit a high degree of large-scale ordering. The method of scanning tunneling microscopy (STM) has proved b be capable of studying the electronic properties of such systems and their structures on a submolecular resolution, level. It was established that, in some systems, H-bonding is the predominant interaction between molecules and governs the molecular self-assembly process.

Selective non-covalent interactions have been widely used in solution chemistry to direct the assembly of molecules into nanometer-sized functional structures such as capsules, switches and prototype nanomachines. The concepts of supramolecular organization have also been applied to two-dimensional (2D) assemblies on surfaces stabilized by means of H-bonding, dipolar coupling, or metal coordination. Another approach to controlling surface structures uses adsorbed molecular monolayers to create preferential binding sites that accommodate individual target molecules. James A. Theobald et al. (Controlling Molecular Deposition and Layer Structure with Supramolecular Surface Assemblies, Nature, 424, 1029-1031 (2003)) combined these approaches by using H-bonding to guide the assembly of two types of molecules into a 2D open honeycomb network. This network controls and templates new surface phases formed by subsequently deposited fullerene molecules. It was found that the open network acts as a 2D array of large pores of sufficient capacity to accommodate several large guest molecules and serves as a template for the formation of an ordered fullerene layer.

The self-assembly of a 2D loosely packed H-bonded network of trimesic acid (TMA) at the liquid-solid interface has been observed using STM (see Lackinger at al., Langmuir, 21, 4984-4988 (2005)). Two crystallographically different 2D phases of TMA were identified and selected by varying the solvent. In this paper, some models of various crystallographic structures with the corresponding H-bonding modes were introduced: (a) chickenwire structure, a=b=1.7 nm, angle [symbol] =60°, area=2.5 nm², 2 molecules per unit cell; (b) flower structure, a=b=2.5 nm, angle [symbol] =60°, area=5.4 nm², 6 molecules per unit cell; (c) “super flower” structure, representing more densely packed 2D TMA polymorph based entirely on 3-fold H-bonding. It was suggested that the denser “flower” structure (b) is likely to be the most thermodynamically stable of the two observed monolayer polymorphs. Studies of these adsorbed polymorph structures for TMA dissolved in a series of acid solvents [CH₃(CH₂)ₙCOOH with n=2−7] showed that the flower structure was favoured for the shorter-chain solvents, which also corresponded to those in which TMA had the maximum solubility. It should be noted that an even more densely packed TMA structure could presumably be formed with a purely 3-fold H-bonded structure (“super flower” structure), but this TMA form was not observed. A possible explanation for this behaviour is the stabilization, in short-chain solvents, of a TMA trimer [(TMA)₃] solution phase nucleation species, which is a likely precursor to the flower form of TMA; however, an explanation based on differential solvent stabilization of the surface monolayer of flower and chickenwire structures cannot be ruled out.

The crystal packing of some fluorinated azobenzenecarboxylic acids was studied by R. Centore and A. Tuzi (Crystal Eng., 6, 87-97. (2003)). The X-ray crystal structures of C₆H₅COOH,C₆F₅COOH (1), C₆H₅CONH₂,C₆F₅CONH₂ (2), and C₆H₅CONH₂,C₆F₅COOH (3) were analyzed in order to elucidate the role of Ph-PhF synthon in directing self-assembly and H-bonding in these cocrystals (see Reddy et al., Crystal Growth & Design, 4, 89-94 (2004)). The strong H-bond donor acidity of C₆F₅COOH and C₆F₅CONH₂ together with mixed stacks of phenyl and perfluorophenyl rings steer acid-acid and amide-amide H-bonding in cocrystals 1 and 2. The acid-amide H-bonding is sufficiently strengthened by donor acidity and acceptor basicity in 3, so that the role of the Ph-PhF synthon is weaker because the aromatic rings stack with lateral offset. The complex C₆H₅COOH,C₆F₅CONH₂ (4) could not be obtained under similar crystallization conditions. The crystal structure of C₆F₅CONH₂ was also determined to compare the molecular conformation and H-bonding with motifs in the cocrystals.

It has been found that 4-hydroxybenzoic acid (1) crystallizes into three crystalline forms: (i) monoclinic from a DMSO solution (1A), (ii) triclinic from a solution in 1:1 DMSO/hot ethyl acetate (1B) and (iii) triclinic from a pyridine solution (1C) (see Jayaraman at al., Crystal Growth & Design, 4, 1403-1409 (2004)). The formation of these pseudopolymorphs and the structural similarity of their packing motifs can be rationalized in terms of few-multipoint solutes-solvent interactions. In all three structures, the crystallographic aspects pertaining to the influence of solvent molecules towards the formation of H-bonded network structures are described. In addition to the strong H-bonds, intermolecular C—H . . . O, C—H . . . π, and π . . . π interactions were found to stabilize the crystal structures.

A series of 4,4-dipyridyl (4,4-DP) derivatives have been prepared and studied using single-crystal X-ray diffraction techniques (see D. E. Lynch et al., Crystal Eng., 2, 137-144 (1999)). The structures had increasing degree of complexity in the overall H-bonded network. The structure of 1 comprises polymeric H-bonded chains of associated 4,4-DP and ICA molecules that propagate through complementary sites on the ICA molecules. The structure of 2 consisted of two parallel polymeric H-bonded chains, each involving associated 4,4-DP and 3-ABA molecules cross-linked through complementary 3-ABA sites. The structure of 3 was an extensive 3-dimensional H-bonded network involving all H-bonded donor and acceptor sites on the constituent molecules. In each case, the positions and directions of the N—H groups were important in determining the final lattice network.

As noted above, in a most general case the biaxial film is characterized by three different principal values of the refractive indices n_{A}, n_{B}, n_{C} and the principal axes A, B, C are arbitrary oriented with respect to the laboratory xyz-frame, for which the xy-plane coincides with the optical film plane. Below in the description of the present invention, an important particular case will be used in which the principal axes are oriented in the following way: A∥x; B∥y; C∥z.

The present invention uses in-plane H-bonding applied to a predominantly planar heterocyclic molecular system containing nitrogen hetero-atoms to form a well-ordered planar structure. This idea was checked for heterocyclic compounds substituted with acid residue groups. The experiments have affirmed a possibility of obtaining of the films with desirable optical properties.

In a first aspect of the present invention there is provided an organic compound of the general structural formula (I)

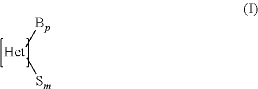

where Het is a predominantly planar heterocyclic molecular system possessing hydrophilic properties; B is a binding group; p is 3, 4, 5, 6, 7 or 8; S is a group providing solubility of the organic compound; m is 0, 1, 2, 3, 4, 5, 6, 7 or 8. The organic compound is transparent for electromagnetic radiation in the visible spectral range from 400 to 700 nm, and a solution of the compound or a salt thereof is capable of forming a substantially transparent optical layer on a substrate, with the heterocyclic molecular planes oriented predominantly parallel to the substrate surface.

In a second aspect of the present invention there is provided an optical film comprising a substrate with front and rear surfaces and at least one solid layer on the substrate, wherein the solid layer comprises at least one organic compound of general structural formula (III)

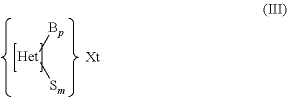

where Het is a predominantly planar heterocyclic molecular system possessing hydrophilic properties; B is a binding group; p is 3, 4, 5, 6, 7, or 8; S is a molecular group providing solubility of the organic compound; m is 0, 1, 2, 3, 4, 5, 6, 7, or 8; X is a counterion from a list comprising H⁺, NH₄⁺, NH(C₂H₅)₃⁺, NH(CH₃)₃⁺, NH(C₃H₇)₃⁺, Na⁺, K⁺, Li⁺, Cs⁺, Ba²⁺, Ca²⁺, Mg²⁺, Sr²⁺, La³⁺, Zn²⁺, Zr⁴⁺, Ce³, Y³⁺, Yb³⁺, Gd³⁺, and any combination thereof; t is the number of counterions for the given organic compound. The heterocyclic molecular planes are oriented predominantly parallel to the surface of said substrate, and said solid layer acid is transparent for electromagnetic radiation in the visible spectral range from 400 to 700 nm. The solid layer is preferably on the front surface of the substrate.

In a third aspect of the present invention there is provided a method for producing an optical film, the method comprising several steps. The first step is preparation of a solution of organic compound of the general structural formula (I) or its salt

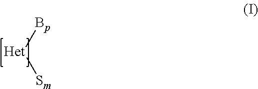

Here, Het is a predominantly planar heterocyclic molecular system possessing hydrophilic properties; B is a binding group; p is 3, 4, 5, 6, 7, or 8; S is a molecular group providing solubility of the organic compound; m is 0, 1, 2, 3, 4, 5, 6, 7, or 8. At least some of said heterocyclic molecular system is capable of forming flat anisometric particles in the solution owing to lateral interaction of the binding groups via noncovalent chemical bonds. The second step is an application of a liquid layer of the solution of the organic compound onto the substrate. The liquid layer is substantially transparent for electromagnetic radiation in the visible spectral range from 400 to 700 nm. The flat anisometric particles of the heterocyclic molecular systems are bound among themselves owing to lateral interaction of the binding groups via non-covalent chemical bonds and are predominantly oriented in the plane of the substrate. The final step is a drying with the formation of a solid layer.

In a fourth aspect of the present invention there is provided a method for the synthesis of 2,2′-bibenzheteroazole derivatives represented by the general structural formula (IV):

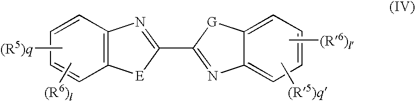

where q+l=0, 1, 2, 3 or 4; q′+l′=0, 1, 2, 3 or 4; E and G moieties are selected independently from the list comprising O, S, NR⁴ where R⁴ is independently selected from the list comprising H, NH₂, OH; R⁵, R′⁵, R⁶ and R′⁶ are substituents selected independently from the list comprising —COOH, —COMe, —CO₂Me, —CONH₂, —CONHNH₂, —SO₃H, —SO₂NH₂, —SO₂—NH—SO₂—NH₂, which method comprises following steps:
a) reacting a component of formula (V) with a component selected from the list comprising structures (VII) and (VIII);
b) reacting the compound obtained in the previous step with a component of formula (VI);

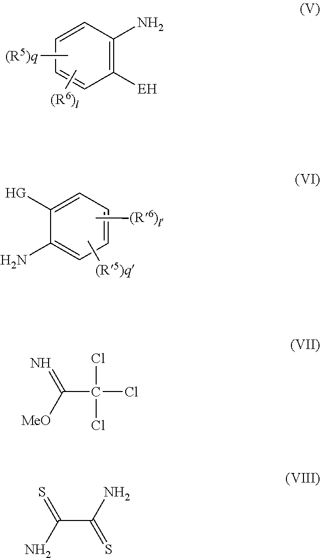

wherein the solvent is selected from the list comprising AcOH, DMF, MeOH, EtOH and mixtures thereof.

In a fifth aspect of the present invention there is provided a method for the synthesis of 2,2′-bibenzheteroazole derivatives represented by the general structural formula (IV′):

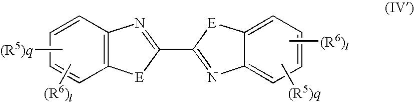

where q+l=0, 1, 2, 3 or 4; E moiety is selected from the list comprising O, S, NR⁴ where R⁴ is selected from the list comprising H, NH₂, OH; R⁵ and R⁶ are substituents selected independently from the list comprising —COOH, —COMe, —CO₂Me, —CONH₂, —CONHNH₂, —SO₃H, —SO₂NH₂′, —SO₂—NH—SO₂—NH₂, which method comprises following steps
a) reacting a component of formula (V) with a component selected from the list comprising structures (VII) and (VIII), and

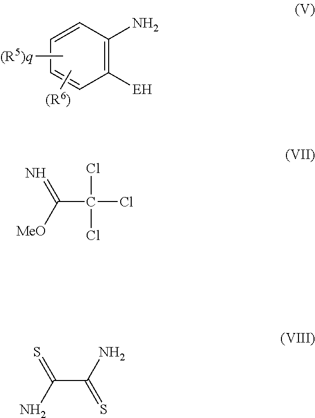

(b) stirring the mixture;
   wherein the solvent is selected from the list comprising AcOH, DMF, MeOH, EtOH and mixtures thereof.

In one embodiment, twice the molar quantity of the component of formula (V) is present in the reaction mixture as compared with the molar quantity of the component selected from the list comprising structures (VII) and (VIII).

In one embodiment of the invention, said method further comprises treating the mixture with Et₃N followed by HCl, wherein the treating action is simultaneous with, or subsequent to, the stirring step.

In a sixth aspect of the present invention there is provided a 2,2′-bibenzheteroazole derivative of the general structural formula (IV)

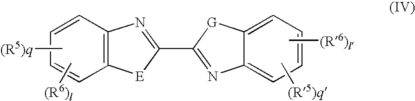

where q+l=0, 1, 2, 3 or 4; q′+=0, 1, 2, 3 or 4; E and G moieties are selected independently from the list comprising O, S, NR⁴ where R⁴ is selected from the list comprising H, NH₂, OH; R⁵, R′⁵, R⁶ and R′⁶ are substituents selected independently from the list comprising —COOH, —COMe, —CO₂Me, —CONH₂, —CONHNH₂, —SO₃H, —SO₂NH₂, and —SO₂—NH—SO₂—NH₂.

In a seventh aspect of the present invention there is provided a method of synthesis of a tricarboxy-5,11,17-trimethyl-11,17-dihydro-5H-bisbenzimidazo[1′,2′:3,4;1″,2″:5,6][1,3,5]triazino[1,2-a]benzimidazole-6,12,18-triium bromide represented by the general structural formula (IX):

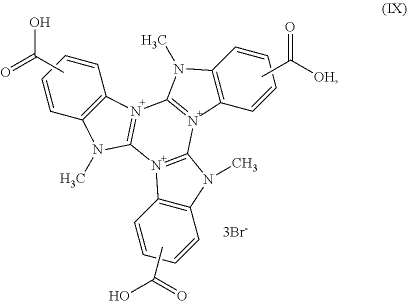

comprising the steps of:
a) bromination and methylation of 1H-benzimidazole-6-carboxylic acid, and
b) condensation of the 2-bromo-1-methyl-1H-benzimidazole-5(6)-carboxylic acids obtained in step (a).

In an eighth aspect of the present invention there is provided a method of synthesis of a bisbenzimidazo[1′,2′:3,4;1″,2″:5,6][1,3,5]triazino[1,2-a]benzimidazole-tricarboxylic acid represented by the general structural formula (X):

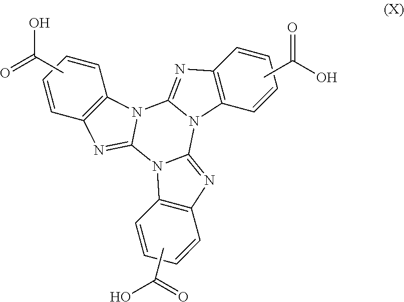

comprising the steps of:
a) preparation of methyl 3,4-diaminobenzoate dihydrochloride comprising the step of bubbling hydrogen chloride through a solution of 3,4-diaminobenzoic acid in methanol;
b) preparation of methyl 2-oxo-2,3-dihydro-1H-benzimidazole-5-carboxylate by condensation of methyl 3,4-diaminobenzoate dihydrochloride from step (a) with urea;
c) transformation of methyl 2-oxo-2,3-dihydro-1H-benzimidazole-5-carboxylate from step (b) to methyl 2-chloro-1H-benzimidazole-5(6)-carboxylate by treatment with hydrogen chloride and phosphorus oxychloride;
d) preparation of trimethyl bisbenzimidazo[1′,2′:3,4;1″,2″:5,6][1,3,5]triazino[1,2-a]benzimidazole-tricarboxylates by trimerization of obtained methyl 2-chloro-1H-benzimidazole-5(6)-carboxylate from step (c); and
e) alkaline hydrolysis of methyl esters of trimethyl bisbenzimidazo[1′,2′:3,4;1″,2″:5,6][1,3,5]triazino[1,2-a]benzimidazole-tricarboxylates from step (d) to obtain product (X).

The general description of the present invention having been made, a further understanding can be obtained by reference to the specific preferred embodiments, which are given herein only for the purpose of illustration and are not intended to limit the scope of the appended claims.

The present invention relates to the creation of organic compounds suitable for manufacturing optical films in which the molecular planes are oriented predominantly parallel to the surface of substrate.

The structure of the disclosed organic compounds is preferably characterized by the presence of three or more binding groups selected from a list comprising hetero-atoms, COO⁻, SO₃⁻, HPO₃⁻, PO₃²⁻, NH, NH₂, CO, OH, NHR, NR, COOMe, CONH₂, CONHNH₂, SO₂NH₂, —SO₂—NH—SO₂—NH₂ and any combination thereof, where radical R is alkyl or aryl that enables lateral H-bonding of heterocyclic molecules and their aggregates with each other with the tendency to the formation of planar H-bonded supramolecules. Preferred alkyl and aryl groups are listed below:

### Alkyl Groups:

General formula: CₙH₂ₙ₊₁— where n is 1 to 23, and is preferably 1, 2, 3 or 4
Examples: Methyl (CH₃—), Ethyl (C₂H₅—), Propyl (CH₃CH₂CH₂— or CH₃CH(CH₃)—), Butyl (CH₃CH₂CH₂CH₂— or C(CH₃)₃— or CH₃CH₂CH(CH₃)— or CH₃CH(CH₃)CH₂—)

### Aryl-Groups:

### Examples: Phenyl (C₆H₅—), Benzyl (C₇H₇—), Naphthyl (C₁₀H₇—)

Said acid groups provide for the physical adsorption of selected heterocyclic compounds on various substrates, comprising those made of carbon, diamond, gold, silver, glass, and many other materials. The interaction of the hydrophilic surface with the system of H-bonds formed by binding groups in planar supramolecules may induce the in-plane orientation of the supramolecules. The hydrophilic surface and planar H-bonded supramolecules form layers on the substrate surface.

The arrangement of acid groups influences the structure of planar H-bonded supramolecules and may produce various structural motifs with different spatial structures.

The organic compounds of the general structural formula (I) can be prepared using any conventional method known in prior art. Some heterocyclic compounds can be synthesized by cyclization of fragments containing carboxylic groups or by introducing substituents into commercially available heterocyclic systems, with their subsequent modification.

In order to obtain an optical film containing planar H-bonded heterocyclic molecules oriented parallel to the substrate, it is preferable to provide for the interactions of two types in this system:

The first factor is the interaction (adsorption) of planar heterocyclic molecules (adsorbate) with the substrate (adsorbent) that results in the desired orientation of molecules or their aggregates at the substrate surface. The adsorption of molecules can be either physical (physisorption) or chemical (chemisorption). The physical adsorption is mediated by intermolecular forces and is not accompanied by significant changes in the electron structure of adsorbed molecules. In this case, the adsorbed molecules (admolecules) usually retain surface (lateral) mobility. The chemical adsorption involves the formation of chemical bonds between molecules of the adsorbate and adsorbent. Thus, chemisorption can be considered as a kind of chemical reaction in a region confined to the surface layer of the adsorbate. Obviously, the chemical bonds limit the surface mobility of admolecules. The disclosed invention employs combinations of organic compounds (adsorbates) and substrates featuring predominantly physical adsorption. Therefore, the adsorbed molecules and their aggregates can move over the substrate surface.

Second, the physically adsorbed planar heterocyclic molecules should interact with each other by means of weak lateral forces acting in the substrate plane. These intermolecular forces play an important role in the formation of a long-range order in the adlayer and in the final single layer. The lateral interaction can be provided by H-bonds formed between some substituents of the heterocyclic molecules.

In one embodiment of an organic compound according to this invention, the predominantly planar heterocyclic molecular system is a partially or completely conjugated. In another embodiment of an organic compound according to this invention, the heterocyclic molecular system comprises hetero-atoms selected from the list comprising nitrogen, oxygen, sulfur, and any combination thereof. In one embodiment of an organic compound according to this invention, at least one of the binding groups is selected from the list comprising said hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is alkyl or aryl, as disclosed hereinabove. In still another embodiment of the disclosed organic compound, at least one of the binding groups is complementary group. Identical binding groups belonging to different heterocyclic molecular systems may form noncovalent chemical bonds between these systems. Such binding groups are called self-binding or complementary. In other embodiment of an organic compound according to this invention, at least one of the binding groups is selected from the list comprising hydrogen acceptor (A), hydrogen donor (D), and group having the general structural formula (II)

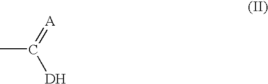

wherein the hydrogen acceptor (A) and hydrogen donor (D) are independently selected from the list comprising NH-group, and oxygen (O).

In one embodiment of the disclosed invention, the organic compound further ensures the absorption of electromagnetic radiation in at least one predetermined subrange of the UV spectral range.

In another embodiment of the disclosed invention, at least one of the binding groups serves as a group providing solubility of the organic compound in water or in organic solvents. The groups S providing solubility of the organic compound in water may be selected from the list consisting of COOH, SO₃H, H₂PO₃ and any combination thereof. The groups S providing solubility of the heterocyclic molecular system in organic solvents may be selected from the list consisting of CONR¹R², CONHCONH₂, SO₂NR¹R², R³ or any combination thereof, wherein R¹, R² and R³ are selected from hydrogen, alkyl, and aryl, as defined hereinabove.

In one embodiment of the disclosed invention, the heterocyclic molecular system has an extended anisometric form having longitudinal axis. In another embodiment of the disclosed invention, the heterocyclic molecular system has an axis of symmetry of order k (Cₖ) directed perpendicularly with respect to the plane of heterocyclic molecular system, where k is an integer of no less than 3. Examples of predominantly planar heterocyclic molecular systems with pyrazine or/and imidazole fragments having a general structural formula are shown in the Table 1.

TABLE 1
Examples of predominantly planar heterocyclic molecular systems with pyrazine or/and imidazole fragments

| | |
|---|---|
| 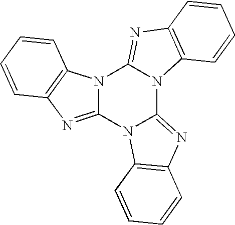 | (1) |
| 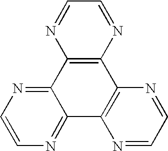 | (2) |
| 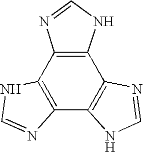 | (3) |
| 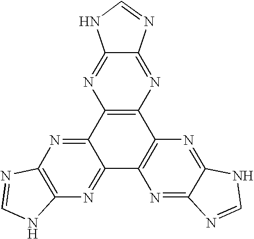 | (4) |

In one embodiment of the organic compound, the heterocyclic molecular system (Het) has general structural formulas shown in the Table 2 where E and G moieties are selected independently from the list comprising O, S, and NR⁴ (where R⁴ is selected from the list comprising H, NH₂, OH):

TABLE 2
Examples of predominantly planar heterocyclic molecular systems having
an extended anisometric form

| | |
|---|---|
| 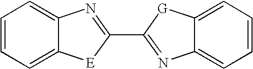 | (5) |
| 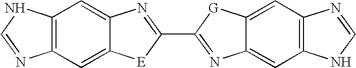 | (6) |

In one embodiment of the organic compound, the heterocyclic molecular system is an oligomer comprising imidazole or/and benzimidazole cycles, which are capable of forming hydrogen bonds. Examples of predominantly linear heterocyclic molecular systems with the oligomer comprising imidazole or/and benzimidazole cycles having a general structural formula are shown in the Table 3, where n is a number in the range from 1 to 5.

TABLE 3
Examples of heterocyclic molecular systems containing oligomer
comprising imidazole or/and benzimidazole cycles

| | |
|---|---|
| 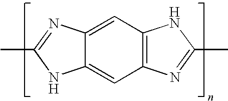 | (7) |
| 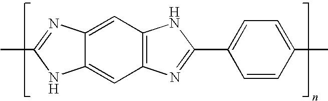 | (8) |
| 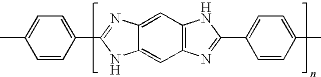 | (9) |
| 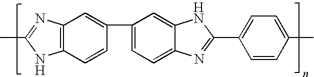 | (10) |
| 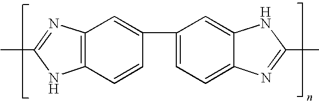 | (11) |
| 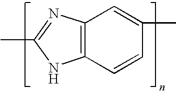 | (12) |
| 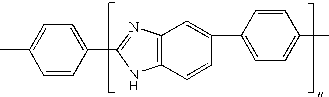 | (13) |
| 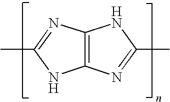 | (14) |
| 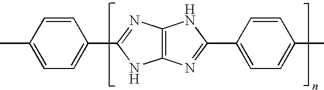 | (15) |

In another embodiment of the present invention, the organic compound is selected from the list comprising derivatives of 1H,1′H-2,2′-bibenzimidazole, derivatives of 2,2′-bi-1,3-benzoxazole, and derivatives of 2,2′-bi-1,3-benzothiazole. In yet another embodiment of the present invention, the organic compound has general structural formulas shown in the Table 4.

TABLE 4
Examples of 2,2′-bibenzheteroazole derivatives

| | |
|---|---|
| 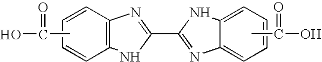 | (16) |
| 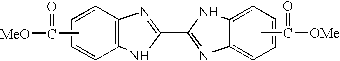 | (17) |
| 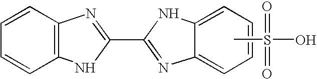 | (18) |
| 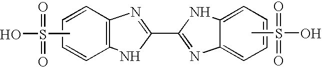 | (19) |
| 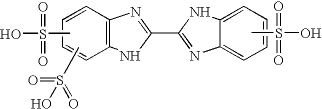 | (20) |
| 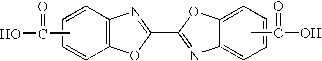 | (21) |
| 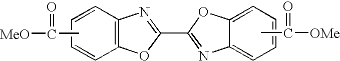 | (22) |
| 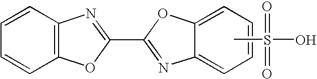 | (23) |
| 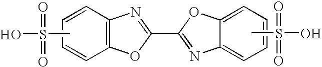 | (24) |
| 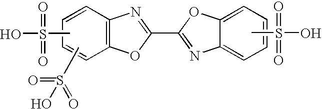 | (25) |
| 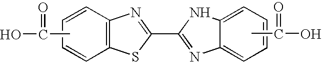 | (26) |
| 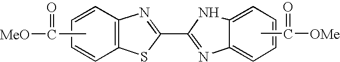 | (27) |
| 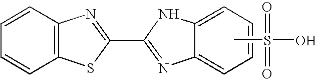 | (28) |
| 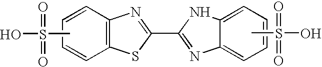 | (29) |
| 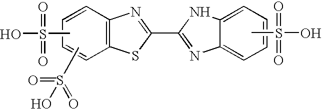 | (30) |
| 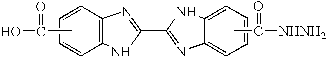 | (31) |
| 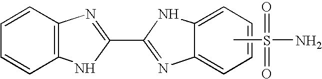 | (32) |
| 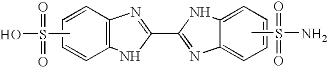 | (33) |
| 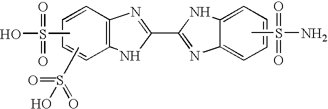 | (34) |

In another embodiment of the organic compound, the binding groups provide formation of flat anisometric particles in the solution via non-covalent chemical bonds. In still another embodiment of the organic compound, the non-covalent chemical bond is selected from the list comprising single hydrogen bond, dipole-dipole interaction, cation—pi-interaction, Van-der-Waals interaction, coordination bond, ionic bond, ion-dipole interaction, multiple hydrogen bond, interaction via the hetero-atoms and any combination thereof. In yet another embodiment of the organic compound, at least one binding group provides a labile equilibrium of anisometric particles within the solution. In one embodiment of present invention, the organic compound further comprises at least one additional substituent selected from a list comprising —CH₃, —C₂H₅, —NO₂, —Cl, —Br, —F, —CF₃, —CN, —NCS, —OH, —OCH₃, —OC₂H₅, —OCOCH₃, —OCN, —SCN, —NH₂, —NHCOCH₃, and —CONH₂.

The present invention also provides an optical film as disclosed hereinabove. In one embodiment of the disclosed optical film, the predominantly planar heterocyclic molecular system is partially or completely conjugated. In another embodiment of the disclosed optical film, said heterocyclic molecular system comprises heteroatoms selected from the list comprising nitrogen, oxygen, sulfur, and any combination thereof. In still another embodiment of the disclosed optical film, at least one binding group is selected from the list comprising hydrogen acceptor (A), hydrogen donor (D), and group having the general structural formula (II)

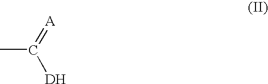

wherein the hydrogen acceptor (A) and hydrogen donor (D) are independently selected from the list comprising NH-group, and oxygen (O). In one embodiment of the disclosed optical film, the binding group is selected from the list comprising said hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is alkyl or aryl, as disclosed hereinabove. In another embodiment of the disclosed optical film, at least one binding group is a complementary group. In still another embodiment of the disclosed optical film, said organic layer absorbs electromagnetic radiation in at least one predetermined wavelength subrange of the UV spectral range. In another embodiment of the optical film, the binding groups provide formation via non-covalent chemical bonds of flat anisometric particles in the solid layer, which are predominantly oriented in plane of substrate surface. In one embodiment of the disclosed optical film, the non-covalent chemical bond is selected from the list comprising single hydrogen bond, dipole-dipole interaction, cation—pi-interaction, Van-der-Waals interaction, coordination bond, ionic bond, ion-dipole interaction, multiple hydrogen bond, interaction via the hetero-atoms and any combination thereof.

In one embodiment of the optical film, said organic layer is substantially insoluble in water. In another embodiment of the optical film the organic layer comprises two or more organic compounds of the general structural formula (III) each ensuring the absorption of electromagnetic radiation in at least one predetermined wavelength subrange of the UV spectral range.

In one embodiment of the disclosed optical film, the heterocyclic molecular system has the axis of symmetry of order k (Cₖ) directed perpendicularly with respect to the plane of molecular system, where k is an integer of no less than 3.

In another embodiment of the disclosed optical film, the heterocyclic molecular system has an extended anisometric form having longitudinal axis. In yet another embodiment of the disclosed optical film, the longitudinal axes of the heterocyclic molecular systems have approximately isotropic alignment in the substrate plane. In still another embodiment of the disclosed optical film, the longitudinal axes of the heterocyclic molecular systems have approximately anisotropic alignment in the substrate plane.

In one embodiment of the disclosed optical film, the predominantly planar heterocyclic molecular system comprises pyrazine or/and imidazole fragments. Examples of those planar conjugated heterocyclic systems are given in Table 1. In yet another embodiment of the disclosed optical film, the heterocyclic molecular system has an extended anisometric form shown in Table 2. In yet another embodiment of the disclosed optical film, the heterocyclic molecular system is an oligomer comprising imidazole or/and benzimidazole cycles, which are capable of forming hydrogen bonds. Examples of those planar conjugated heterocyclic systems are given in Table 3, where n is a number in the range from 1 to 5. In still another embodiment of the disclosed optical film, the organic compound is selected from the list comprising derivatives of 1H,1′H-2,2′-bibenzimidazole, derivatives of 2,2′-bi-1,3-benzoxazole, and derivatives of 2,2′-bi-1,3-benzothiazole. In yet another embodiment of the optical film, the organic compound has a general structural formula shown in the Table 4. In one embodiment of the disclosed optical film, the organic compound further comprises at least one additional substituent selected from a list comprising —CH₃, —C₂H₅, —NO₂, —Cl, —Br, —F, —CF₃, —CN, —NCS, —OH, —OCH₃, —OC₂H₅, —OCOCH₃, —OCN, —SCN, —NH₂, —NHCOCH₃, and —CONH₂.

In one embodiment of the optical film, the longitudinal axes of the heterocyclic molecular systems have approximately isotropic alignment in the substrate plane. In another embodiment of the optical film, the longitudinal axes of the heterocyclic molecular systems have approximately anisotropic alignment in the substrate plane.

In still another embodiment of the optical film, said solid layer is generally a uniaxial retardation layer possessing two refraction indices (nx and by) corresponding to two mutually perpendicular directions in the plane of the substrate surface and one refraction index (nz) in the normal direction to the substrate surface, and wherein the refractive indices obey the following condition: nx=ny>nz. In yet another embodiment of the disclosed optical film, said solid layer is generally a biaxial retardation layer possessing one refraction index (nz) in the normal direction to the substrate surface and two refraction indices (nx and ny) corresponding to two mutually perpendicular directions in the plane of the substrate surface and wherein the refractive indices obey the following condition: nx>ny>nz. In still another embodiment of the disclosed optical film, the substrate is made of a polymer. In yet another embodiment of the disclosed optical film, the substrate is made of a glass. In one embodiment of the disclosed optical film, the rear surface of the substrate is covered with an antireflection or antiflashing coating. In one embodiment of the disclosed optical film, the substrate is transparent for electromagnetic radiation in the visible spectral range. In another embodiment, the present invention provides an optical film wherein the transmission coefficients of the substrate in the visible spectral range are not less than 90%. In still another embodiment, the optical film further comprises an additional planarization transparent layer applied onto the front surface of the substrate. In another embodiment of the disclosed invention the optical film comprises a reflective layer applied onto the rear surface of the substrate. In one embodiment of the disclosed optical film, the substrate is a specular or diffusive reflector. In still another embodiment of the disclosed optical film, the substrate is a reflective polarizer. In still another embodiment, the present invention provides an optical film further comprising an additional transparent adhesive layer applied on top of the organic layer. In another embodiment of the disclosed invention the optical film further comprises a protective coating applied on the transparent adhesive layer. In one embodiment of the disclosed invention, the optical film comprises two or more organic layers, wherein these layers comprise different organic compounds of the general structural formula (III) ensuring the absorption of electromagnetic radiation in at least one independently selected wavelength subrange of the UV spectral range. In another embodiment of the disclosed optical film, the solid layer is partially or entirely crystal layer. In another embodiment of the disclosed invention, the optical film comprises two or more organic layers, wherein these layers comprise different organic compounds of the general structural formula (III) ensuring a difference at least one of refraction indices (nx, ny, or nz) in two adjacent layers.

The present invention also provides a method for producing an optical film, as disclosed hereinabove. In one embodiment of the disclosed method, the predominantly planar heterocyclic molecular system is partially or completely conjugated. In another embodiment of the disclosed method, the heterocyclic molecular system further comprises at least one additional substituent selected from a list comprising —CH₃, —C₂H₅, —NO₂, —Cl, —Br, —F, —CF₃, —CN, —NCS, —OH, —OCH₃, —OC₂H₅, —OCOCH₃, —OCN, —SCN, —NH₂, —NHCOCH₃, and —CONH₂. In yet another embodiment of the disclosed method, the binding groups are selected from the list comprising hydrogen acceptor (A), hydrogen donor (D), and group having the general structural formula (II)

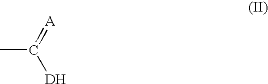

wherein the hydrogen acceptor (A) and hydrogen donor (D) are independently selected from the list comprising NH-group and oxygen (O). In still another embodiment of the disclosed method, the heterocyclic molecular system comprises hetero-atoms selected from the list comprising nitrogen, oxygen, sulfur, and any combination thereof. In one embodiment of the disclosed method, at least one of the binding groups is selected from the list comprising the hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is alkyl or aryl. In one embodiment of the disclosed invention, identical binding groups belonging to different heterocyclic molecular systems form noncovalent chemical bonds between these systems. Such binding groups are called self-binding or complementary. In one embodiment of the method, the liquid layer ensures the absorption of electromagnetic radiation in at least one predetermined wavelength subrange of the UV spectral range. In another embodiment of the disclosed method, the heterocyclic molecular system has an extended anisometric form having longitudinal axis. Isotropic or anisotropic alignment of the longitudinal axes of the heterocyclic molecular systems may be received for the same organic compound. In yet another embodiment of the disclosed method, a concentration of the solution, a level of temperature, humidity and duration of the drying step may be selected so as to ensure approximately isotropic alignment of the longitudinal axes of the heterocyclic molecular systems in the substrate plane. In still another embodiment of the disclosed method, a concentration of the solution, a level of temperature, humidity and duration of the drying step and characteristics of an alignment action may be selected so as to ensure approximately anisotropic alignment of the longitudinal axes of the heterocyclic molecular systems in the substrate plane. In one embodiment of the disclosed method, the heterocyclic molecular system has the axis of symmetry Cₖ directed perpendicularly with respect to the plane of molecular system, where n is the number no less than 3. In another embodiment of the method, the predominantly planar heterocyclic molecular system comprises pyrazine or/and imidazole fragments and has a general structural formula from the list comprising structures 1-4 shown in Table 1. In yet another embodiment of the method, the predominantly planar heterocyclic molecular system has an extended anisometric form shown in Table 2.

In one embodiment of the disclosed method, the heterocyclic molecular system is an oligomer comprising imidazole or/and benzimidazole cycles, which are capable of forming hydrogen bonds. Said heterocyclic molecular systems may have the general structural formulas from the list comprising structures 7-15 shown in Table 3, where n is a number in the range from 1 to 5. In still another embodiment of the disclosed method, the organic compound is selected from the list comprising derivatives of 1H,1′H-2,2′-bibenzimidazole, derivatives of 2,2′-bi-1,3-benzoxazole, and derivatives of 2,2′-bi-1,3-benzothiazole. In yet another embodiment of the disclosed method, the organic compound has a general structural formula shown in the Table 4. In another embodiment of the disclosed method, the non-covalent chemical bond is selected from the list comprising single hydrogen bond, dipole-dipole interaction, cation—pi-interaction, Van-der-Waals interaction, coordination bond, ionic bond, ion-dipole interaction, multiple hydrogen bond, interaction via the hetero-atoms and any combination thereof. In still another embodiment of the method, said liquid layer further comprises a solvent selected from the list comprising water, water-miscible solvent, alcohol-based solvent, and any combination thereof. In one embodiment of the disclosed method, the preferred solvent is water. In yet another embodiment of the method, the drying is executed in airflow. In still another embodiment, the amount of solvent is controlled so as to provide the liquid-layer viscosity necessary for applying a liquid layer by means of a hydrodynamical flow. In yet another embodiment of the disclosed method, the liquid-layer viscosity does not exceed 2 Pa·s. In another embodiment of the invention, the method further comprises a pretreatment step before the application onto the substrate. In one embodiment of the disclosed method, the pretreatment comprises the step of making the surface of the substrate hydrophilic. In another embodiment of the disclosed method, the pretreatment further comprises application of a planarization layer. In one embodiment of the disclosed invention, the method further comprises a post-treatment step with solution of any aqueous-soluble inorganic salt with a cation selected from the list containing H⁺, Ba²⁺, Ca²⁺, Mg²⁺, Sr²⁺, La³⁺, Zn²⁺, Zr⁴⁺, Ce³⁺, Y³⁺, Yb³⁺, Gd³⁺, and any combination thereof. In one embodiment of the disclosed method, the application and post-treatment steps are carried out simultaneously. In another embodiment of the disclosed method, the drying and post-treatment steps are carried out simultaneously. In still another embodiment of the disclosed method, the post-treatment step is carried out after drying. In yet another embodiment of the disclosed method, the application is made using an isotropic solution. In still another embodiment of the disclosed method, the application is made using a lyotropic liquid crystal solution. In one embodiment of the disclosed method, the liquid layer made of a gel. In one embodiment of the disclosed method, the application is made using a viscous liquid phase. In yet another embodiment of the disclosed invention, the method after the application further comprises an alignment action applied onto said liquid layer on the substrate. In one embodiment of the disclosed method, the sequence of technological operations of the deposition and the drying is repeated two or more times and each consequent solid layer is formed using a liquid layer, this liquid layer made of organic compound being either the same or different from that used in the previous cycle and having an absorption of electromagnetic radiation in at least one independently selected wavelength subrange of the UV spectral range. In one embodiment of the disclosed invention, the method further comprises an alignment action applied onto said liquid layer on the substrate after or simultaneously with the application step being the different from that used in the previously cycle and having the difference at least one of refraction indices (nx, ny, or nz) in two adjacent layers.

The present invention also provides a 2,2′-bibenzheterdazole heterocyclic compound as disclosed hereinabove. In one embodiment of the present invention, the 2,2′-bibenzheteroazole heterocyclic compound has the general structural formula from the list comprising structures 16 to 34 shown in Table 4.

Other objects and advantages of the present invention will become apparent upon reading the detailed description of the examples and the appended claims provided below, and upon reference to the drawings, in which:

FIGS. 1 to 3 are described hereinabove.

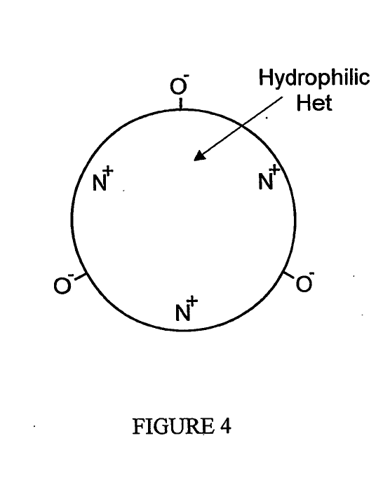

FIG. 4 schematically shows an organic compound comprising hydrophilic disk-like heterocyclic molecular system (Het) with three binding groups.

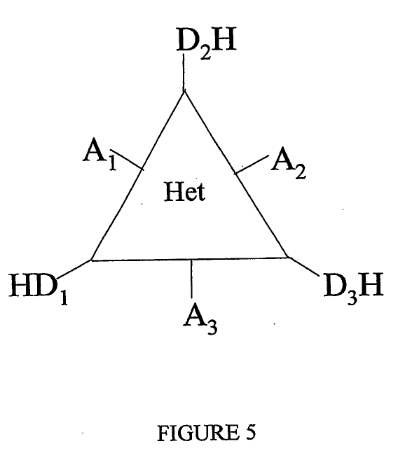

FIG. 5 schematically shows organic compound comprising a flat heterocyclic molecular system having an axis of symmetry C₃.

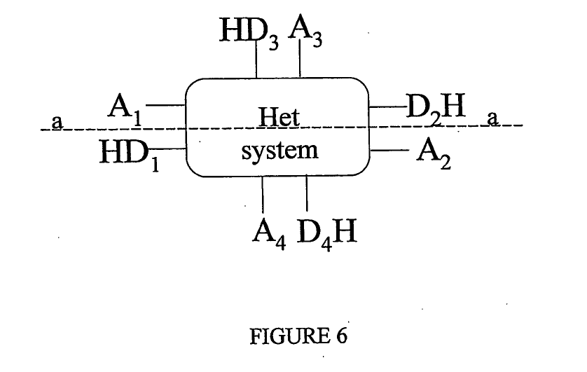

FIG. 6 schematically shows organic compound comprising a flat heterocyclic molecular system having an extended anisometric form having longitudinal axis.

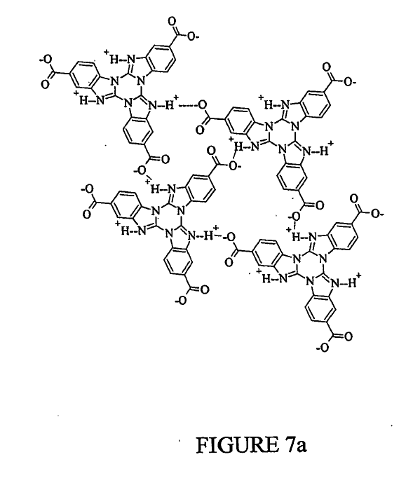

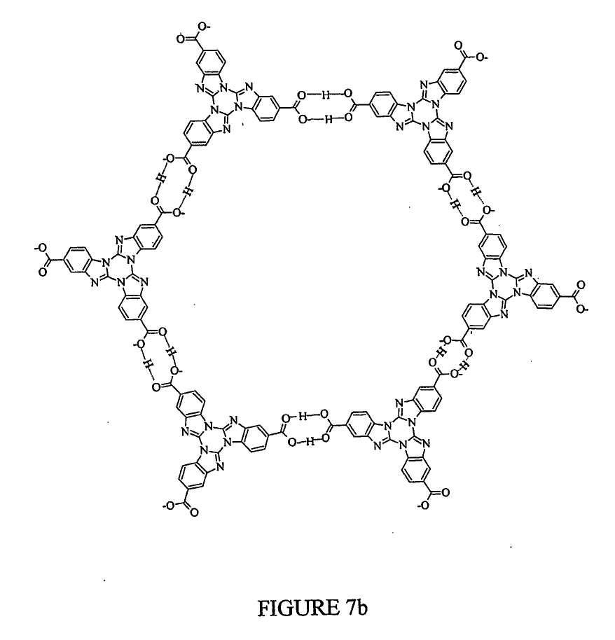

FIG. 7a shows one structure of a flat anisometric particle formed by the heterocyclic molecular systems shown in Table 1 as structure 1.

FIG. 7b shows another structure of a flat anisometric particle formed by the heterocyclic molecular systems shown in Table 1 as structure 1.

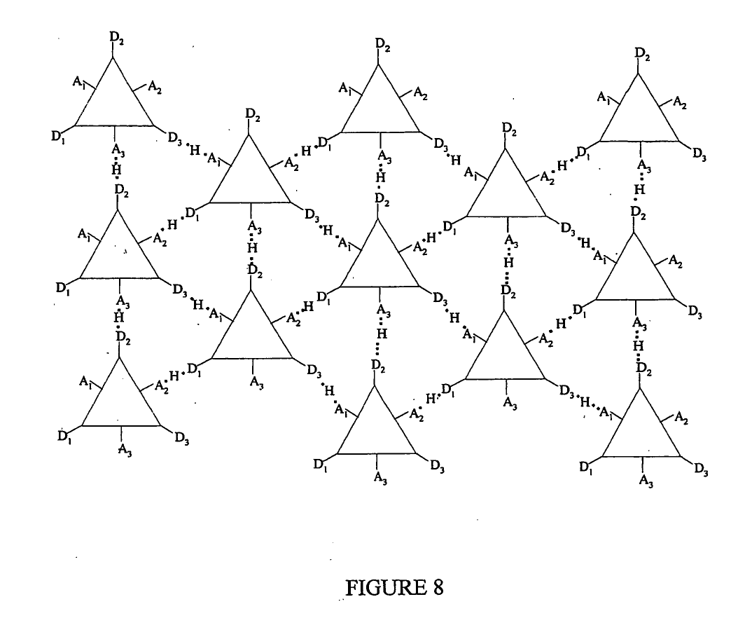

FIG. 8 schematically shows one possible structure of flat anisometric particles formed by organic compound having the general structural formula shown in FIG. 5.

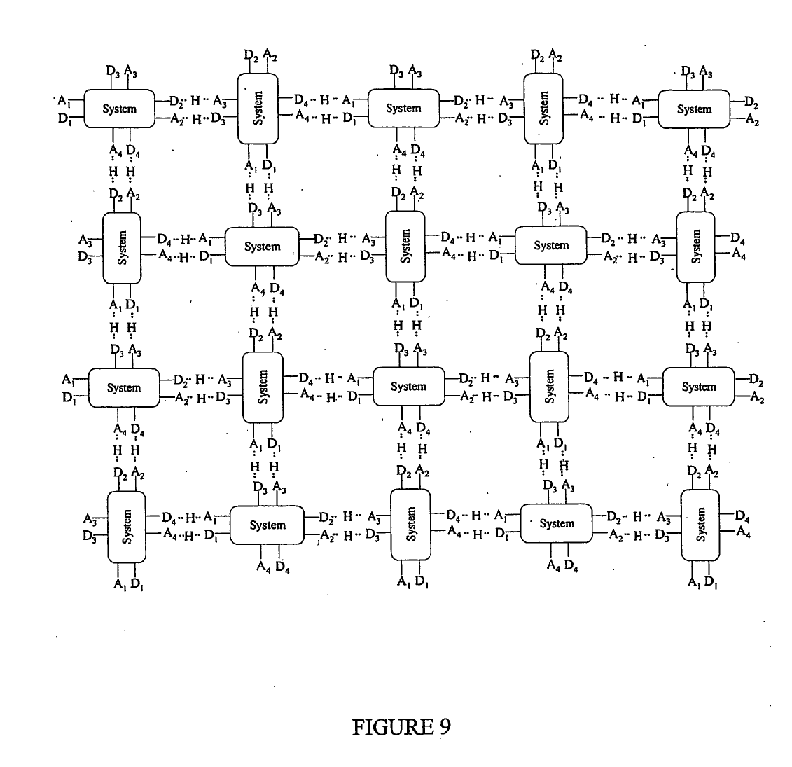

FIG. 9 schematically shows another possible structure of flat anisometric particles formed by organic compound having the general structural formula shown in FIG. 6.

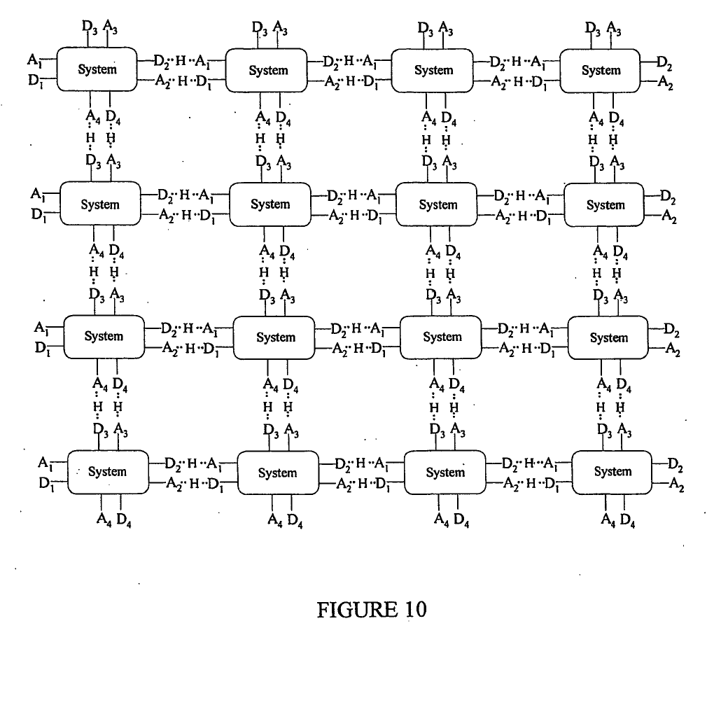

FIG. 10 schematically shows yet another possible structure of flat anisometric particles formed by organic compound having the general structural formula shown in FIG. 6.

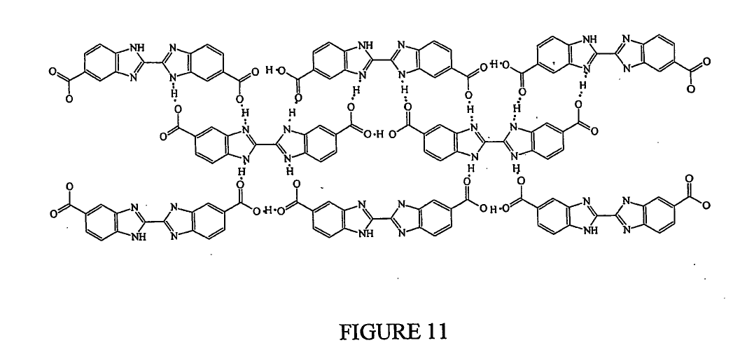

FIG. 11 shows a structure of a flat anisometric particle formed by the heterocyclic molecular systems shown in Table 2 as structure 5.

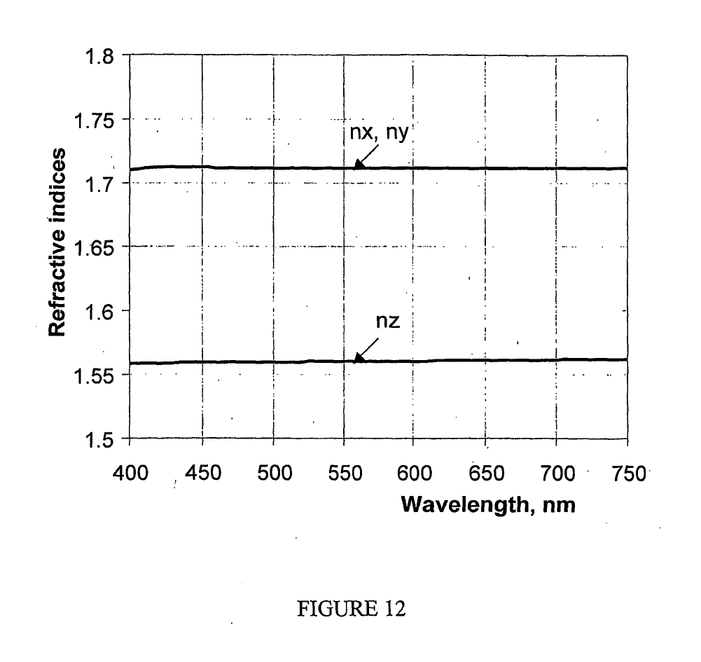

FIG. 12 shows refractive indices spectra of optical film according to present invention.

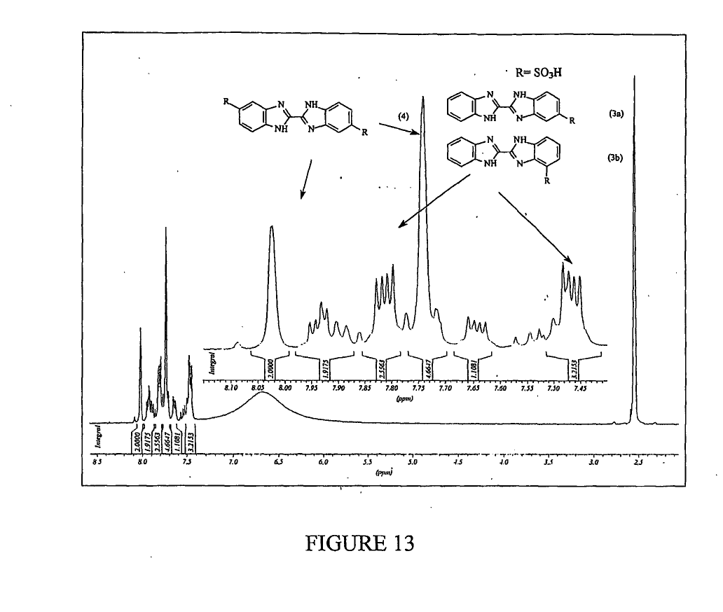

FIG. 13 shows NMR¹ H spectrum (Brucker Avance 300 instrument, d₆-dimethyl sulfoxide).

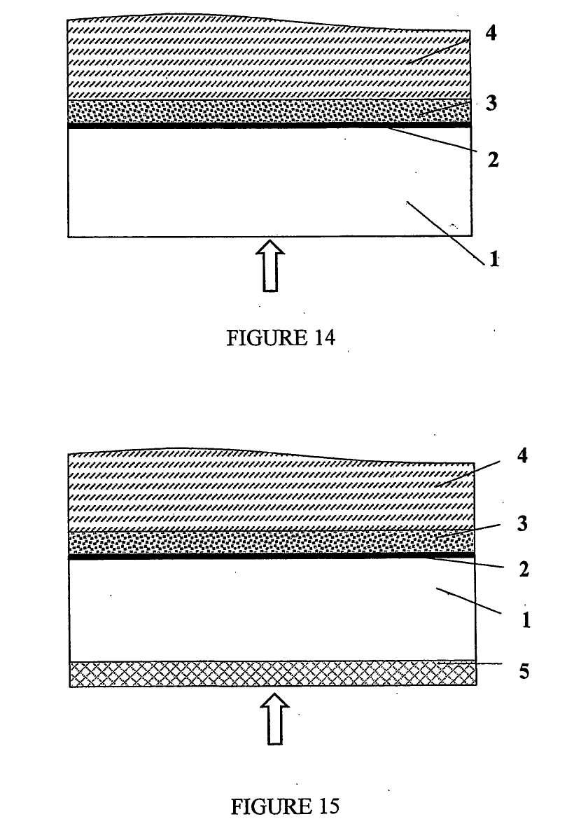

FIG. 14 shows a section of an optical film on a substrate, together with additional adhesive and protective layers.

FIG. 15 shows a section of an optical film with an additional antireflection layer.

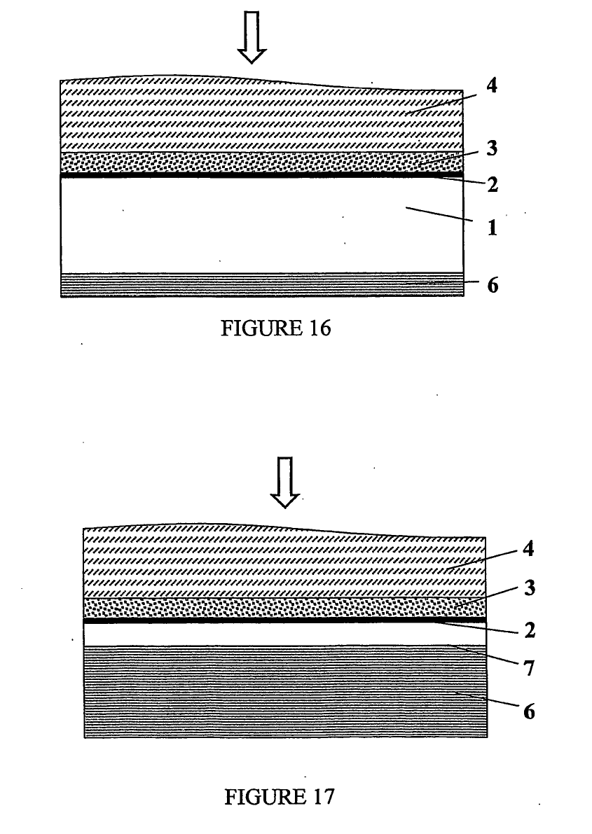

FIG. 16 shows a section of an optical film with an additional reflective layer.

FIG. 17 shows a section of an optical film with a diffuse or specular reflector as the substrate.

FIG. 4 schematically shows one embodiment of organic compound comprising a flat disk-like heterocyclic molecular system and at least three binding groups. The heterocyclic molecular system having the general structural formula I shown in the Table 1 may be used as such disk-like heterocyclic molecular system. The positions of binding groups are indicated by oxygen ions (O⁻). The heterocyclic molecular system has the third-order axis of symmetry directed perpendicularly to its plane. The given heterocyclic molecular systems contain nitrogen cations (N⁺) acting as heteroatoms. As a result, electric dipoles (O⁻—N⁺) are formed in the plane of the heterocyclic molecular system, which impart hydrophilic properties to the system. In the course of preparation of a solution of organic compound, the heterocyclic molecular systems and binding groups form flat anisometric particles (kinetic particles) due to noncovalent chemical bonds between the binding groups of adjacent heterocyclic molecular systems. During application of the reaction mixture onto a substrate, a certain fraction of these flat anisometric particles are destroyed because of the rupture of weak noncovalent bonds. This destruction reduces viscosity of the reaction mixture and facilitates its orientation by the hydrodynamic flow. The planes of anisometric particles are oriented parallel to the substrate plane (due to the hydrophilic properties of the heterocyclic molecular systems, which produces their effective homeotropic alignment. Then, the ruptured noncovalent chemical bonds in the anisometric particle's are restored.

FIG. 5 schematically shows one embodiment of organic compound comprising a flat heterocyclic molecular system having an axis of symmetry C₃ directed perpendicularly with respect to the plane of heterocyclic molecular system. The heterocyclic molecular system comprises binding groups of two types. The binding groups denoted as A₁, A₂ and A₃ are capable to form hydrogen bonds (H-bonds) and serve as hydrogen acceptors. Other binding groups (D₁H, D₂H and D₃H) are capable to form hydrogen bonds (H-bonds) also and serve as hydrogen donor. In the course of preparation of a solution of organic compound, the heterocyclic molecular systems and the binding groups form flat anisometric particles (kinetic particles) due to H-bonds between the binding groups of adjacent heterocyclic molecular systems.

FIG. 6 schematically shows one embodiment of organic compound comprising a flat heterocyclic molecular system having an extended anisometric form having longitudinal axis (a-a). The heterocyclic molecular system comprises binding groups of two types. The binding groups denoted as A₁, A₂, A₃ and A₄ are capable to form hydrogen bonds (H-bonds) and serve as hydrogen acceptors. Other binding groups (D₁H, D₂H, D₃H and D₄H) are capable to form hydrogen bonds (H-bonds) also and serve as hydrogen donor. In the course of preparation of a solution of organic compound, the heterocyclic molecular systems and the binding groups form flat anisometric particles (kinetic particles) due to H-bonds between the binding groups of adjacent heterocyclic molecular systems.

FIGS. 7a and 7b schematically show several possible structures of flat anisometric particles (polymer particles). In this embodiment of the disclosed invention, noncovalent bonds are formed between cations (N⁺) of one heterocyclic molecular system and anions (O⁻) of the adjacent heterocyclic molecular systems (see FIG. 7a). If the binding groups are represented by carboxy groups, then H-bonds are formed between hydroxy group OH of one carboxy group and oxygen ion of another group (see FIG. 7b). The dimensions of anisometric particles preferably do not exceed one micron.

FIG. 8 schematically shows one possible structure of flat anisometric particles (polymer particles) formed by organic compound having the general structural formula shown in FIG. 5. In this embodiment of the disclosed invention, the flat anisometric particles are formed due to following H-bonds: A₁-H-D₃, A₂-H-D₁, and A₃-H-D₂. FIG. 9 schematically shows another possible structure of flat anisometric particles (polymer particles) formed by organic compound having the general structural formula shown in FIG. 6. Characteristic feature of these flat anisometric particles is approximately perpendicular orientation of longitudinal axes of the adjacent planar heterocyclic molecular systems. FIG. 10 schematically shows yet another possible structure of flat anisometric particles (polymer particles) formed by organic compound having the general structural formula shown in FIG. 6. Characteristic feature of these flat anisometric particles is approximately parallel orientation of longitudinal axes of the adjacent planar heterocyclic molecular systems. In this embodiment of the disclosed invention, the flat anisometric particles are formed due to following H-bonds: A₁-H-D₂, A₂-H-D₁, A₃-H-D₄, and A₄-H-D₃.

In another embodiment of the present invention, the optical film is based on an orgariic compound containing the heterocyclic molecular system shown in Table 2 as structure 5, which exhibits elongated ribbon-like configuration, possesses hydrophilic properties, and has two terminal carboxylic groups as binding groups. In the solution of organic compound, such molecular systems form isometric particles having the configuration shown in FIG. 11. When the solution of the organic compound is applied onto a substrate, for example, by extrusion, said isometric particles and, hence, binding groups are oriented along the coating direction (Ox). FIG. 7 schematically shows one embodiment, in which the isometric particle consists of linear chains with the binding groups capable of forming H-bonds. Owing to the hydrophilic properties of heterocyclic molecular systems, their planes are oriented parallel to the substrate (xOy plane). The optical film according to this embodiment is anisotropic and its refraction indices are substantially different along the three axes: nx>ny>nz.

In order that the invention may be more readily understood, reference is made to the following examples, which are intended to be illustrative of the invention, but are not intended to be limiting in scope.

### EXAMPLE 1

The first example describes syntheses of the mixture of tricarboxy-5,11,17-trimethylbis[3,1]benzimidazo[1′,2′:3,4;1″,2″:5,6][1,3,5]triazino[1,2-a][3,1]benzimidazole-5,11,17-triium bromides:

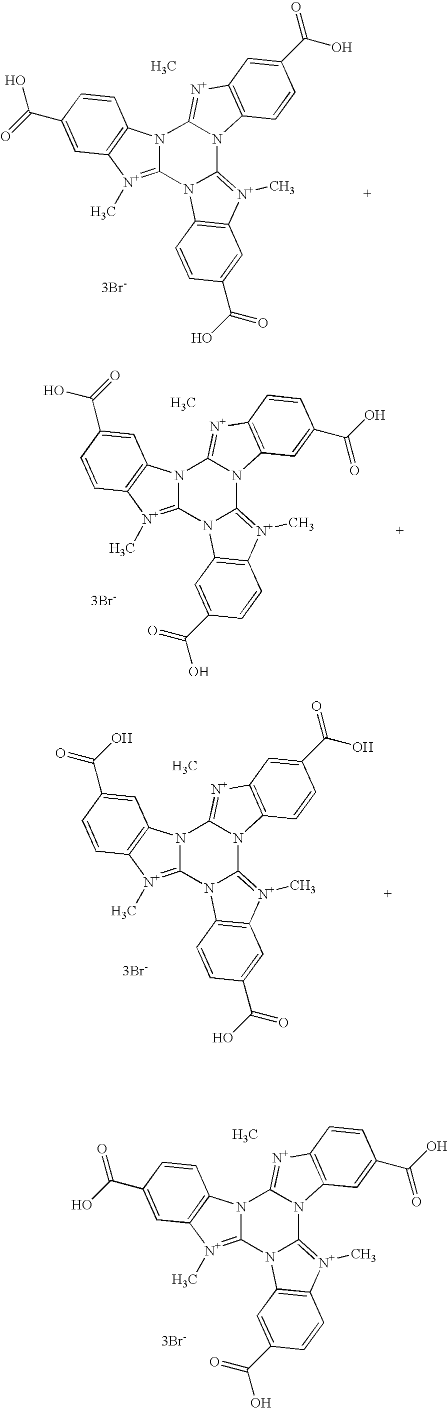

### A. Synthesis of 1H-benzimidazole-6-carboxylic acid

3,4-Diaminobenzoic acid (20 g, 0.13 mol) was suspended in Formic acid (120 ml) while cooling. After that Hydrochloric acid (12 ml) was added. Obtained reaction mass was agitated for 24 hours at the room temperature. Reaction mixture was filtered through fiber glass filter. Then filter cake was dissolved in water (400 ml), pH was adjusted to 2.0 with ammonia solution and reaction mass was agitated overnight. Resulting suspension was filtered. pH of filtrate was adjusted to 3.0 with ammonia solution and reaction mass was agitated for two hours. Precipitate was filtered and dried at ˜100° C. H¹NMR (Brucker Avance-300, DMSO-d₆, δ, ppm): 7.695 (d, H, CH^{Ar}(7)); 7.877 (dd, H, CH^{Ar}(6)); 8.25 (d, H, CH^{Ar}(4)); 8.44 (s, H, (s, H, CH^{Ar}(2)); 12.82 (s, 2H, NH, COOH). Yield 11.3 g (54%).

### B. Synthesis of 2-bromo-1-methyl-1H-benzimidazole-5(6)-carboxylic acids

Solution of Bromine (64 ml, 1.25 mol) in Methanol (200 ml) was charged into suspension of 1H-benzimidazole-6-carboxylic acid (20 g, 0.12 mol) in methanol (270 ml) with cooling. Reaction mass was agitated for three days at the room temperature. After that solution volume was reduced down to 120 ml on a rotary evaporator. Obtained concentrate was added to acetone (1.7 l) and agitated overnight. Precipitate was filtered, rinsed with acetone and dried. H¹NMR (Brucker Avance-300, DMSO-d₆, δ, ppm): 3.94 (s, 3H, CH₃); 7.995 (d, H, CH^{Ar}(7)); 8.14 (dd, H, CH^{Ar}(6)); 8.39 (d, H, CH^{Ar}(4)); 9.79 (s, H, COOH). Yield 20.5 g (65%).

### C. Synthesis of tricarboxy-5,11,17-trimethylbis[3,1]benzimidazo[1′,2′:3,4;1″,2″:5,6][1,3,5]triazino[1,2-a][3,1]benzimidazole-5,11,17-triium bromides

2-Bromo-1-methyl-1H-benzimidazole-5(6)-carboxylic acids (20 g, 0.08 mol) was charged into N-methylpyrrolidone (100 ml). Reaction mass was agitated for 6 hours at 150-155° C. After self cooling it was added to chloroform (2 l) and agitated for two hours. Precipitate was filtered. Filter cake was suspended in chloroform (700 ml), filtered and rinsed with chloroform. The product was dried at ˜90° C.

H¹NMR (Brucker Avance-300, DMSO-d₆, δ, ppm): 4.12 (s, 3H, CH₃); 4.14 (s, 3H, CH₃); 4.16 (s, 3H, CH₃); 4.18 (s, 3H, CH₃); 8.105 (m, 6H, 3*CH^{Ar}CH^{Ar}); 8.485 (m, 3H, 3*CH^{Ar}); 9.90 (s, H, COOH^{Ar}); 9.705 (m, 3H, COOH). The product was dried at ˜90° C. Yield 18.8 g (90%).

### EXAMPLE 2

The second example describes syntheses of the mixture of bisbenzimidazo[1′,2′:3,4;1″,2″:5,6][1,3,5]triazino[1,2-a]benzimidazole-trisulfonic acids:

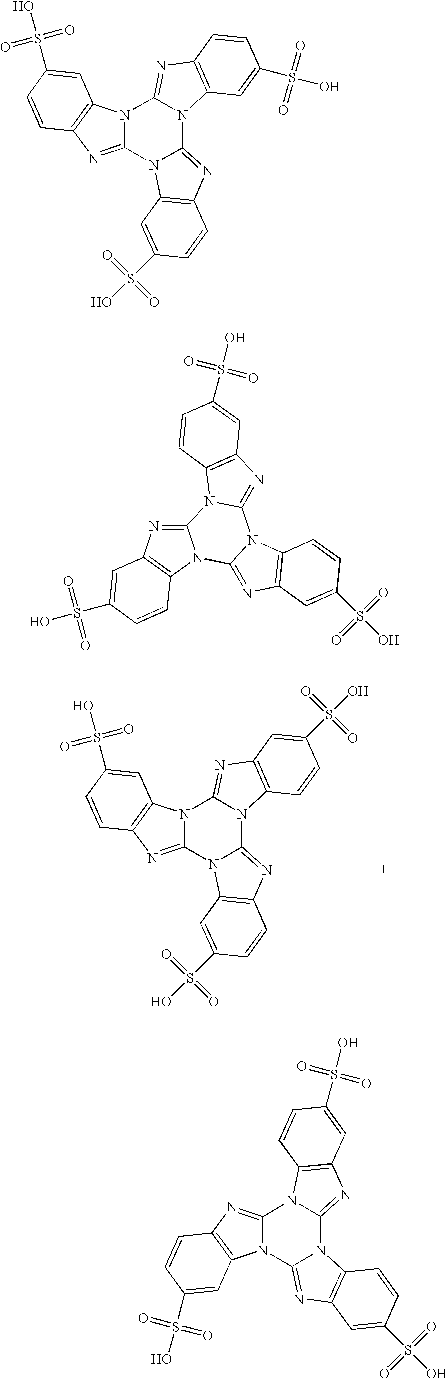

### A. Synthesis of bisbenzimidazo[1′,2′:3,4;1″,2″:5,6][1,3,5]triazino[1,2-a]benzimidazole

2-Chloro-1H-Benzimidazole (4 g, 0.026 mol) was heated up to 200-220° C. and agitated for half hour (until hydrogen chloride stopped to evolve). Nitrobenzene was added into reaction mass and boiled for 25 minutes with agitation. After self codling down to ˜80° C. it was filtered and rinsed with acetone. Filter cake was dried at ˜100° C. Yield 2.1 g (70%).

### B. Synthesis of the mixture of bisbenzimidazo[1′,2′:3,4;1″,2″:5,6][1,3,5]triazino[1,2-a]benzimidazole-trisulfonic acids

Bisbenzimidazo[1′,2′:3,4;1″,2″:5,6][1,3,5]triazino[1,2-a]benzimidazole (2.0 g, 0.006 mol) was charged into 20% oleum (20 ml) and agitated overnight. After that reaction mass was diluted with water (28.2 ml). Precipitate was filtered and rinsed with concentrated hydrochloric acid, 1,4-dioxane and acetone. The product was dried in desiccator. Yield 1.32 g (40%).

### EXAMPLE 3

### A056

The next example describes the preparation of an optical film from a solution of heterocyclic compound.

7.5 g of mixture of tricarboxy-5,11,17-trimethylbis[3,1]benzimidazo[1′,2′:3,4;1″,2″:5,6][1,3,5]triazino[1,2-a][3,1]benzimidazole-5,11,17-triium bromides obtained in the Example 1 is dissolved in 42.5 g deionized water and stirred at 20° C. until total dissolution of the solid phase and then 21.3 g of a 5% ZnCl₂ solution and 28.7 g deionized water is added and the mixture is stirred for 1 hr under ambient conditions.

The soda-lime LCD quality glass slides are prepared for coating by treating in a 10% NaOH solution for 30 min, rinsing with deionized water, and drying in airflow with the aid of a compressor. Prior to the coating, samples are rinsed with isopropyl alcohol. The obtained solution is applied onto a glass plate with a Mayer rod #2.5 at a temperature 20° C. and relative humidity of 65%. The film is dried at the same humidity and temperature in gentle airflow.

The refractive indices spectra of the obtained film are presented in FIG. 12. The obtained film is optically isotropic in the plane and exhibits high retardation in the vertical direction.

### EXAMPLE 4

The example describes syntheses of 2,2′-bibenzheteroazole heterocyclic compounds represented by the general structural formula (IV).

1H,1′H-2,2′-bibenzimidazole-5,5′-dicarboxylic acid (1)

O-methyl-1,1,1-trichloroacetimidate was added (0.4 mL, 0.57 g, 3.2 mmol) to a suspension of 3,4-diaminobenzoic acid (1.0 g, 6.6 mmol) in anhydrous methanol (100 mL). The reaction mixture was stirred for 48 h at ambient conditions. Resultant yellow solid material was filtered off, dried in vacuum to a constant weight. Yield 0.43 g (41%). For further purification 1H,1′H-2,2′-bibenzimidazole-5,5′-dicarboxylic acid was dissolved in dimethylsulfoxide taken in a ratio of 0.85 g/37 mL and water was added slowly (5 mL) to resultant solution. The mixture was stirred for 30 min., solid material formed was filtered off, washed with ethanol (2×30 mL) and dried in vacuum to a constant weight. NMR¹ H spectrum (Brucker Avance 600 instrument; solvent d₆-dimethyl sulfoxide; δ, ppm; J, Hz): 7.74 d.d (2H^{b}, ³J_{ba}=7.5), 7.93 d (2H^{a}, ³J_{ab}=7.5), 8.28 d (2H^{x}), 12.89 br.s (2NH and 2COOH), 13.94 br.s (2NH and 2COOH). Mass-spectrum (MALDI positive mode, Ultraflex TOF/TOF Bruker Daltonics instrument): 322 (100%) [M^{+•}], 304 (45%) [M^{+•}-H₂O], 277 (50%) [M^{+•}-CO₂H].

### Dimethyl 1H,1′H-2,2′-bibenzimidazole-5,5′-dicarboxylate (2)

Methyl 3,4-diaminobenzoate (3.3 g, 19.9 mmol) was dissolved in anhydrous MeOH (100 mL). O-Methyl-1,1,1-trichloroacetimidat (1.75 g, 1.25 mL, 9.9 mmol) was added to the resultant solution. Reaction mixture was stirred for 48 h at ambient conditions. A precipitate formed was filtered off, washed with methanol (2×20 mL) and dried in vacuum to a constant weight. Yield 1.0 g (28%).

### 1H,1′H-2,2′-bibenzimidazole-5,5′-disulfonic acid (4)

A round-bottom 3 neck flask was charged with 3,4-diaminobenzenesulfonic acid (8.0 g, 42.5 mmol) and anhydrous MeOH (0.85 L). O-Methyl-1,1,1-trichloroacetimidat was added (2.8 mL, 3.74 g, 21.2 mmol). The resultant suspension was stirred for 24 h at ambient conditions. Additional amount of O-methyl-1,1,1-trichloroacetimidat was added (1.4 mL, 1.87 g, 10.5 mmol) after this time, then reaction mixture was stirred for 72 h days at ambient conditions, heated for 3 h at 50° C. and triethylamine (14 mL, 9.4 g, 93.5 mmol) was added. Stirring was continued at this temperature for 18 h. Then reaction mixture was cooled to 30° C., and an intensive flow of dry HCl was passed through the solution until a precipitate formed. The suspension was filtered off at 40° C., precipitate was washed with MeOH (4×150 mL, stirring of suspension for 10-15 min each turn) and with MeOH—HCl 3.5% solution (100 mL, 1 h of stirring). Product 1H,1′H-2,2′-bibenzimidazole-5,5′-disulfonic acid was pale yellow or colorless, weight 3.5 g, yield 42%. It may contain own hydrochloride as a salt. NMR¹ H spectrum (Brucker Avance 300 instrument; solvent d₆-dimethyl sulfoxide; δ, ppm; J, Hz): 5.27 br.s (—SO₃H in exchange with H₂O and NH) 7.73 m (2H^{a},2H^{b}), 8.01 br.s (2H^{x}). NMR ¹³C{¹H} spectrum (Brucker Avance 300 instrument; solvent d₆-dimethyl sulfoxide; δ, ppm): 113.00, 115.41, 123.27, 136.44, 137.60, 142.24, 145.34.

### Mixture of 1H,1′H-2,2′-bibenzimidazole-5-sulfonic acid (3a), 1H,1′H-2,2′-bibenzimidazole-4-sulfonic acid (3b), 1H,1′H-2,2′-bibenzimidazole-5,5′-disulfonic acid (4) via direct sulfonation reaction

Bibenzimidazole (0.50 g, 2.1 mmol) was inserted into oleum 20%. Resultant solution was stirred at 30° for 2 h, then was poured into ice (5 g), a white precipitate separated was centrifuged off, a 36% water solution of hydrogen chloride was added (5 mL), a solution formed instantly. This solution was concentrated to ½ of initial volume, diluted with water (5 mL), resultant heterogeneous mixture was cooled to +10° C., precipitate was filtered off and washed with water (5 mL) using a centrifuge, colorless solid material obtained was dried in vacuum yield 0.07 g. The mixture is soluble in 10% HCl. NMR¹ H spectrum (Brucker Avance 300 instrument, d₆-dimethyl sulfoxide) see FIG. 13.

### EXAMPLE 5

The example describes the preparation of an optical film from a water solution of 1H,1′H-2,2′-bibenzimidazole-5,5′-dicarboxylic acid in presence of triethylamine. 1H,1′H-2,2′-bibenzimidazole-5,5′-dicarboxylic acid is synthesized as it is described in the Example 4. Hand coating was performed using rod 1.5HS. Thick films (1-3 μm) were obtained using 20-25% solutions. Such a thickness can follow from higher viscosity of the solution. FIG. 11 schematically shows possible structure of flat anisometric particles (polymer particles). In this embodiment of the disclosed invention, two types of H-bonds are formed: 1) between cations (N⁺) of one heterocyclic molecular system and anions (O⁻) of the adjacent heterocyclic molecular systems and 2) between two atoms of oxygen of the adjacent heterocyclic molecular systems.

### EXAMPLE 6

The example describes syntheses of the mixture of bisbenzimidazo[1′,2′:3,4;1″,2″:5,6][1,3,5]triazino[1,2-a]benzimidazole-tricarboxylic acids:

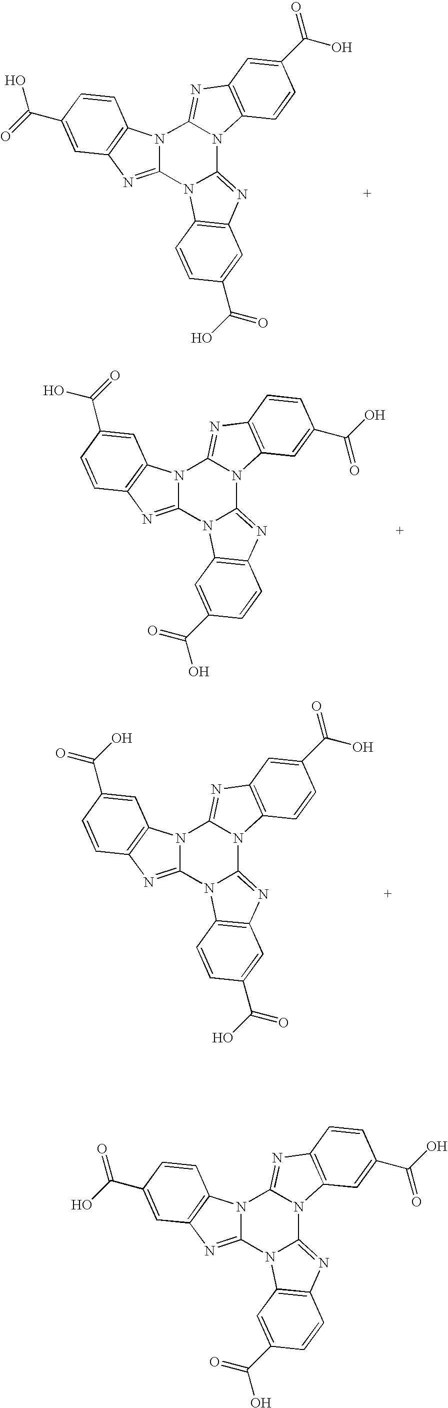

### D. Synthesis of methyl 2-oxo-2,3-dihydro-1H-benzimidazole-6-carboxylate

Methyl 3,4-diaminobenzoate dihydrochloride (20 g, 0.08 mol) was mixed with urea (6.54 g, 0.11 mol). Reaction mixture was heated at ˜150° C. for 7 hours. After cooling powder was suspended in water (400 ml) and pH of the last one was adjusted to 0.45 with hydrochloric acid. Precipitate was filtered and rinsed with water and hydrochloric acid (pH=1.5). Obtained filter cake was dried at ˜100° C. Yield 15.7 g (97%).

### E. Synthesis of methyl 2-chloro-1H-benzimidazole-6-carboxylate

Methyl 2-oxo-2,3-dihydro-1H-benzimidazole-6-carboxylate (43 g, 0.22 mol) was charged into Phosphorus oxychloride (286 ml). Dry hydrogen chloride was bubbled through the boiling reaction mass for 12 hours. After cooling reaction mass was poured in mixture of ice and water (2 kg). Precipitate was filtered out. Filtrate was diluted with water (1.25 l) and ammonia solution (˜800 ml). After that pH was adjusted to 5.6 with ammonia solution. Precipitate was filtered and rinsed with water. Yield 39.5 g (84%).

### F. Synthesis of trimethyl bisbenzimidazo[1′,2′:3,4;1″,2″:5,6][1,3,5]triazino[1,2-a]benzimidazole-tricarboxylates

Methyl 2-chloro-1H-benzimidazole-6-carboxylate (38 g, 0.18 mol) was heated at 185-190° C. for 10 hours. Yield 30.3 g (96%).

### G. Synthesis of bisbenzimidazo[1′,2′:3,4;1″,2″:5,6][1,3,5]triazino[1,2-a]benzimidazoletricarboxylic acids

Trimethyl bisbenzimidazo[1′,2′:3,4;1″,2″:5,6][1,3,5]triazino[1,2-a]benzimidazole-tricarboxylates (30 g, 0.06 mol) was charged into 5% solution of potassium hydroxide (250 ml) and boiled for 1.5 hour. After cooling obtained solution was filtered and neutralized with hydrogen chloride solution. Then pH of solution was adjusted to 1.25 with hydrochloric acid. Precipitate was filtered, rinsed with water and dried at ˜100° C. Mass spectrum (Ultraflex TOF/TOF (Bruker Daltonics, Bremen, Germany)): M/Z=480 (FW=480.39). Yield 26.3 g (95%).

### EXAMPLE 7

FIG. 14 shows the section of an optical film formed on substrate 1. The film contains organic layer 2, adhesive layer 3, and protective layer 4.

### EXAMPLE 8

The above described optical film is applied to the LCD front surface with an additional antireflection layer 5 formed on the substrate (FIG. 15). For example, an antireflection layer of silicon dioxide SiO₂ reduces by 30% the fraction of light reflected from the LCD front surface.

### EXAMPLE 9

With the above described optical film applied to the electrooptical devices or the LCD front surface, an additional reflective layer 6 can be formed on the substrate (FIG. 16). The reflective layer may be obtained, for example, by depositing an aluminium film.

### EXAMPLE 10

The optical film 2 is applied to the diffusive or specular reflector 6 which serves as a substrate (FIG. 17). The reflector layer 6 may be covered with the planarization layer 7 (optional). The planarization layer may be made of polyurethane, or an acrylic polymer, or any other planarized material. The organic layer is manufactured using method described in Example 3. The adhesive layer 3 and the protective layer 4 are applied on top of the optical film.

### EXAMPLE 11

In this example the reflector layer 6 is semitransparent. The organic layer 2 is applied onto the diffuse or specular semitransparent reflector 6 that serves as a substrate (FIG. 17). The reflector layer 6 may be covered with the planarization layer 7 (optional). Polyurethane or an acrylic polymer or any other material may be used for making this planarization layer.

What is claimed is:

1. An organic compound of the general structural formula (I) 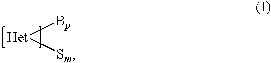
where Het is a predominantly planar heterocyclic molecular system possessing hydrophilic properties;
B is a binding group;
p is 3, 4, 5, 6, 7, or 8;
S is a group providing solubility of the organic compound;
m is 0, 1, 2, 3, 4, 5, 6, 7 or 8;
wherein said organic compound is transparent for electromagnetic radiation in the visible spectral range from 400 to 700 nm, and a solution of the compound or a salt thereof is capable of forming a substantially transparent optical layer on a substrate such that the planes of the heterocyclic molecular systems are oriented predominantly parallel to the substrate plane.

2. An organic compound according to claim 1, wherein the binding groups B provide for the formation of flat particles from the organic compound molecules in solution via non-covalent chemical bonds.

3. An organic compound according to claim 2, wherein at least one binding group B provides a labile equilibrium of the flat particles with the solution.

4. An organic compound according to any of claim 2 or 3, wherein the non-covalent chemical bond is selected from the list comprising a single hydrogen bond, dipole-dipole interaction, cation—pi-interaction, Van-der-Waals interaction, coordination bond, ionic bond, ion-dipole interaction, multiple hydrogen bond, interaction via the hetero-atoms and any combination thereof.

5. An organic compound according to any of claims 1 to 4, wherein at least one binding group B is selected from the list comprising a hydrogen acceptor (A), a hydrogen donor (D), and a group having the general structural formula (II) 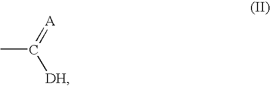
wherein the hydrogen acceptor (A) and hydrogen donor (D) are independently selected from the list comprising NH-group, and oxygen (O).

6. An organic compound according to any of claims 1 to 5, wherein at least one of the binding groups is selected from the list comprising hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is an alkyl group or an aryl group, the alkyl group having the general formula CₙH₂ₙ₊₁— where n is 1 to 23, preferably 1, 2, 3 or 4, the aryl group being selected from the group consisting of phenyl, benzyl and naphthyl.

7. An organic compound according to claim 6, wherein the hetero-atoms are selected from the list, comprising nitrogen, oxygen, sulfur, and any combination thereof.

8. An organic compound according to any of claims 1 to 7, wherein at least one of the binding groups is a complementary group.

9. An organic compound according to any of claims 1 to 8, wherein at least one of the binding groups serves as a group providing solubility of the organic compound in water or in organic solvents.

10. An organic compound according to any of claims 1 to 9, wherein at least one group providing solubility of the organic compound in water is selected from the list comprising COOH, SO₃H, H₂PO₃ and any combination thereof.

11. An organic compound according to any of claims 1 to 9, wherein at least one group providing solubility of the organic compound in organic solvents is selected from the list comprising CONR¹R², CONHCONH₂, SO₂NR¹R², R³, or any combination thereof, wherein R¹, R² and R³ are selected from hydrogen, an alkyl group, an aryl group, and any combination thereof, where the alkyl group has the general formula CₙH₂ₙ₊₁— where n is 1 to 23, and is preferably 1, 2, 3 or 4, and the aryl group is selected from the group consisting of phenyl, benzyl and naphthyl.

12. An organic compound according to any of claims 1 to 11, wherein said predominantly planar heterocyclic molecular system is partially or completely conjugated.

13. An organic compound according to any of claims 1 to 12, wherein the heterocyclic molecular system has an axis of symmetry of order k (Cₖ) directed perpendicularly with respect to the plane of heterocyclic molecular system, where k is an integer of no less than 3.

14. An organic compound according to any of claims 1 to 13, wherein the predominantly planar heterocyclic molecular system comprises pyrazine or/and imidazole cycles, and has a general structural formula selected from the list comprising structures 1-4:

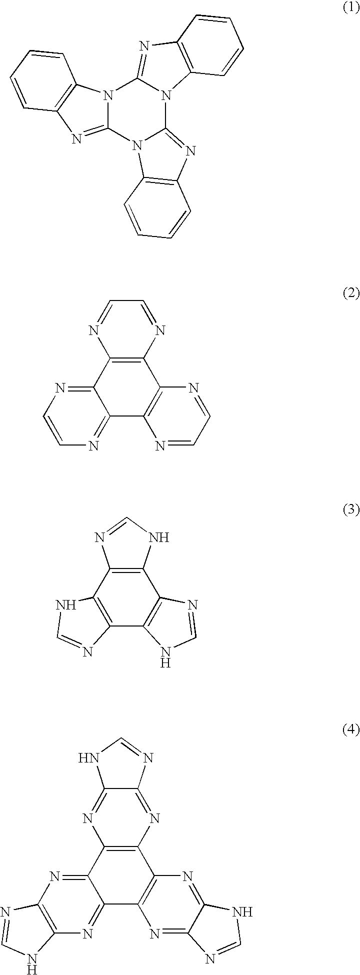

15. An organic compound according to any of claims 1 to 12, wherein the heterocyclic molecular system has an extended anisometric form having a longitudinal axis.

16. An organic compound according to claim 15, wherein the heterocyclic molecular system has a general structural formula selected from structures 5-6, where E and G moieties are selected independently from the list comprising O, S, and NR⁴, where R⁴ is selected from the list comprising H, NH₂, OH:

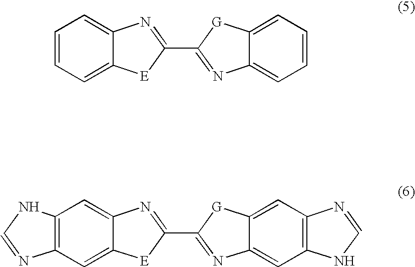

17. An organic compound according to any of claim 1 to 12 or 15, wherein the heterocyclic molecular system is an oligomer comprising imidazole or/and benzimidazole cycles, the imidazole or/and, benzimidazole cycles being capable of forming hydrogen bonds.

18. An organic compound according to claim 17, wherein the heterocyclic molecular system has a general structural formula selected from structures 7-15, where n is a number in the range from 1 to 5:

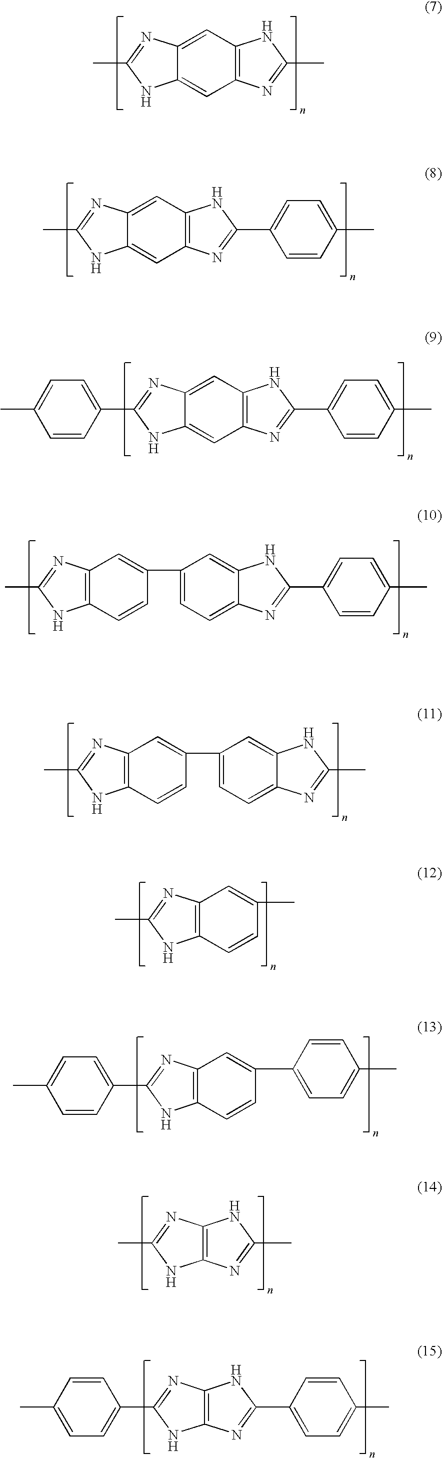

19. An organic compound according to any of claim 1 to 12 or 15 selected from the list comprising derivatives of 1H,1′H-2,2′-bibenzimidazole, derivatives of 2,2′-bi-1,3-benzoxazole, and derivatives of 2,2′-bi-1,3-benzothiazole.

20. An organic compound according to claims 19, having general structural formulas selected from structures 16-34:

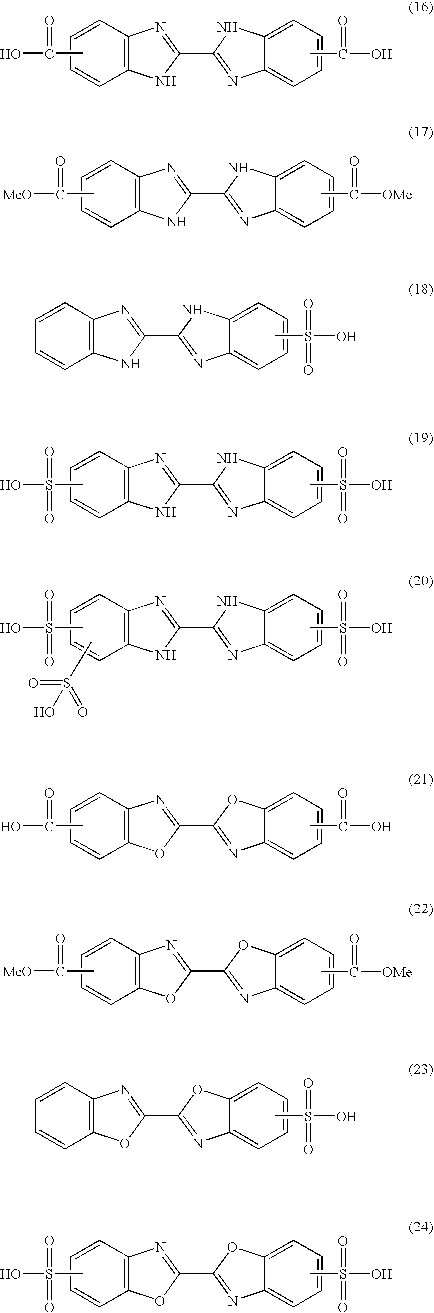

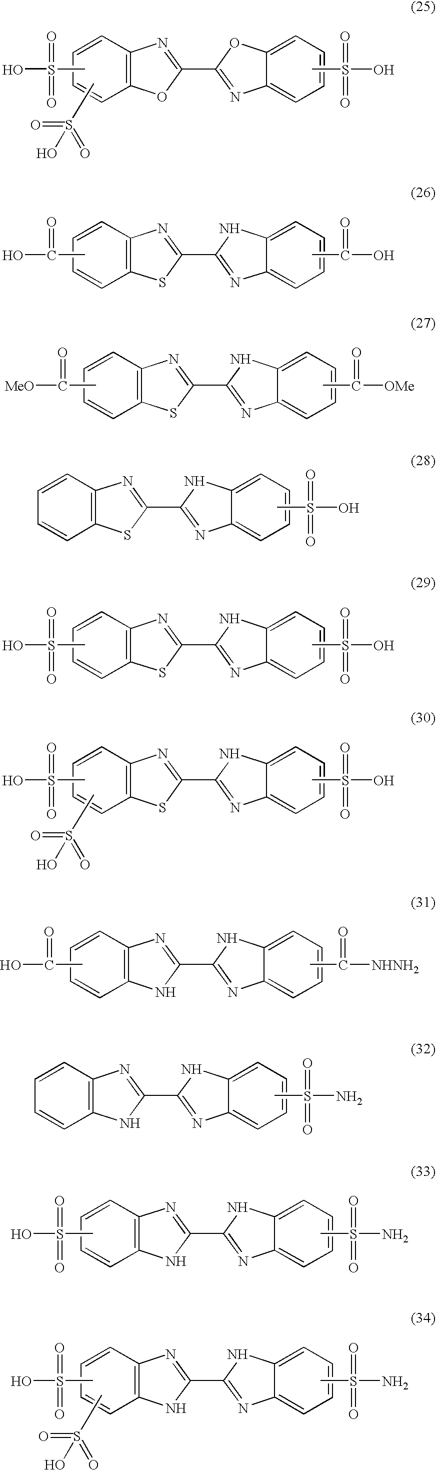

21. An organic compound according to any of claims 1 to 20, further comprising at least one additional substituent selected from a list comprising —CH₃, —C₂H₅, —NO₂, —Cl, —Br, —F, —CF₃, —CN, —NCS, —OH, —OCH₃, —OC₂H₅, —OCOCH₃, —OCN, —SCN, —NH₂, —NHCOCH₃, and —CONH₂.

22. An optical film comprising a substrate having front and rear surfaces and at least one solid layer on the front surface of substrate, wherein the layer comprises at least one organic compound of general structural formula (III) 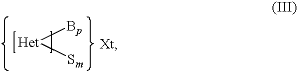
where Het is a predominantly planar heterocyclic molecular system possessing hydrophilic properties;
B is a binding group;
p is 3, 4, 5, 6, 7 or 8;
S is a molecular group providing solubility of the organic compound;
m is 0, 1, 2, 3, 4, 5, 6, 7, or 8;
X is a counterion selected from a list comprising H⁺, NH₄⁺, NH(C₂H₅)₃⁺, NH(CH₃)₃⁺, NH(C₃H₇)₃⁺, Na⁺; K⁺, Li⁺, Cs⁺, Ba²⁺, Ca²⁺, Mg²⁺, Sr²⁺, La³⁺, Zn²⁺, Zr⁴⁺, Ce³⁺, Y³⁺, Yb³⁺, Gd³⁺ and any combination thereof;
t is the number of counterions;
wherein the planes of the heterocyclic molecular systems are oriented predominantly parallel to the substrate surface and said solid layer is transparent for electromagnetic radiation in the visible spectral range from 400 to 700 nm.

23. An optical film according to claim 22, wherein the binding groups provide for the formation via non-covalent chemical bonds of flat anisometric particles in the solid layer, which are predominantly oriented in plane of substrate surface.

24. An optical film according to claim 23, wherein the non-covalent chemical bonds are independently selected from the list comprising a single' hydrogen bond, dipole-dipole interaction, cation—pi-interaction, Van-der-Waals interaction, coordination bond, ionic bond, ion-dipole interaction, multiple hydrogen bond, interaction via the hetero-atoms and any combination thereof.

25. An optical film according to any of claims 22 to 24, wherein the organic compound has at least one binding group selected from the list comprising a hydrogen acceptor (A), a hydrogen donor (D), and a group having the general structural formula (II) 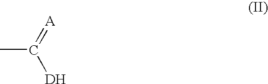
wherein the hydrogen acceptor (A) and hydrogen donor (D) are independently selected from the list comprising NH-group, and oxygen (O).

26. An optical film according to any of claims 22 to 25, wherein the binding group is selected from the list comprising the hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is an alkyl group or an aryl group, the alkyl group having the general formula CₙH₂ₙ₊₁— where n is 1 to 23, preferably 1, 2, 3 or 4, the aryl group being selected from the group consisting of phenyl, benzyl and naphthyl.

27. An optical film according to claim 26, wherein the hetero-atoms are selected from the list comprising nitrogen, oxygen, sulfur, and any combination thereof.

28. An optical film according to any of claims 22 to 27, wherein at least one binding group is complementary group.

29. An optical film according to any of claims 22 to 28, wherein the organic compound further comprises at least one additional substituent selected from a list comprising —CH₃, —C₂H₅, —NO₂, —Cl, —Br, —F, —CF₃, —CN, —NCS, —OH, —OCH₃, —OC₂H₅, —OCOCH₃, —OCN, —SCN, —NH₂, —NHCOCH₃, and —CONH₂.

30. An optical film according to any of claims 22 to 29, wherein said solid layer is substantially insoluble in water.

31. An optical film according to any of claims 22 to 30, wherein said predominantly planar heterocyclic molecular system is partially or completely conjugated.

32. An optical film according to any of claims 22 to 31, wherein the heterocyclic molecular system has an axis of symmetry of order k (Cₖ) directed perpendicularly with respect to the plane of the heterocyclic molecular system, where k is an integer of no less than 3.

33. An optical film according to claim 32, wherein the predominantly planar heterocyclic molecular system comprises pyrazine or/and imidazole cycles and has a general structural formula selected from the list comprising structures 1-4:

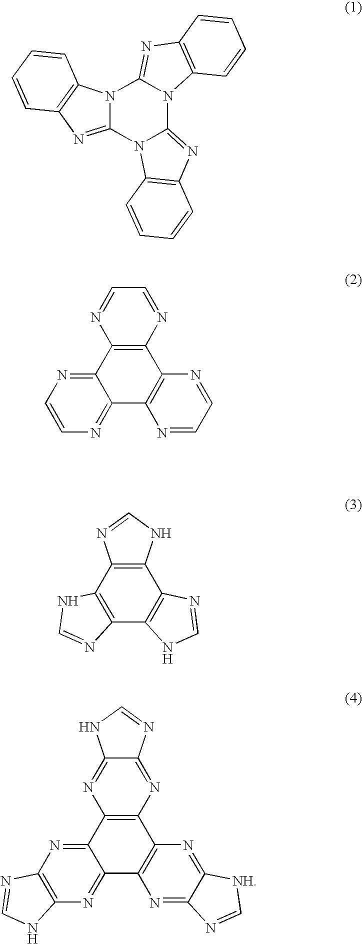

34. An optical film according to any of claims 22 to 31, wherein the heterocyclic molecular system has an extended anisometric form having a longitudinal axis.

35. An optical film according to claim 34, wherein the heterocyclic molecular system has a general structural formula selected from structures 5-6 where E and G moieties are selected independently from the list comprising O, S, and NR⁴ (where R⁴ is selected from the list comprising H, NH₂, OH):

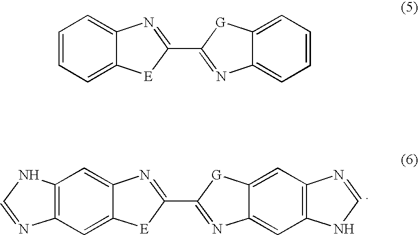

36. An optical film according to any of claim 22 to 31 or 34, wherein the heterocyclic molecular system is an oligomer comprising imidazole or/and benzimidazole cycles, the imidazole or/and benzimidazole cycles being capable of forming hydrogen bonds.

37. An optical film according to claim 36, wherein the heterocyclic molecular system has a general structural formula selected from structures 7-15, where n is a number in the range from 1 to 5:

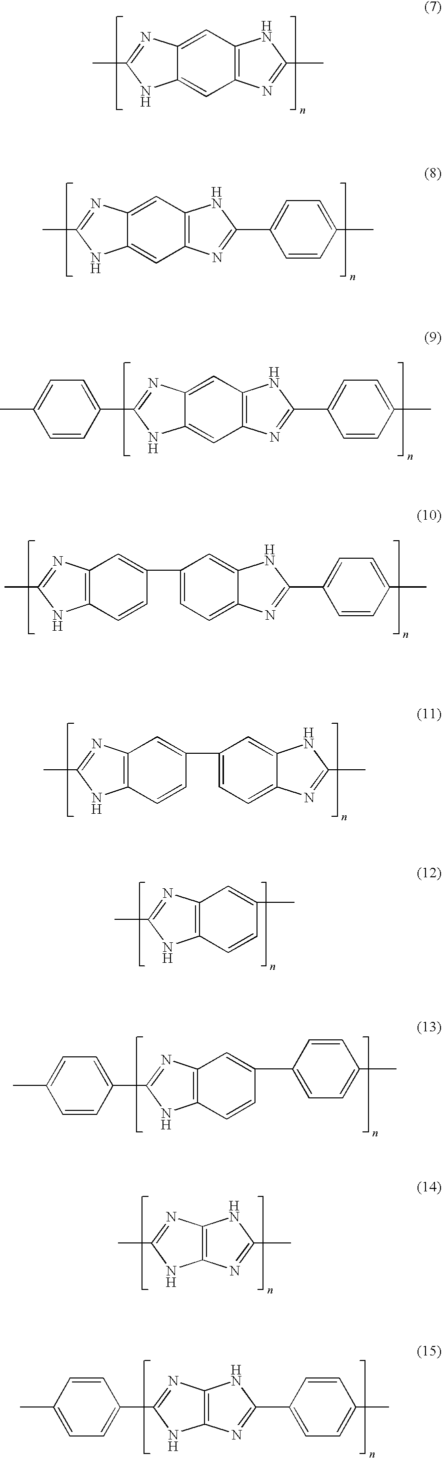

38. An optical film according to any of claims 22 to 31 or 34 to 35, wherein the organic compound is selected from the list comprising derivatives of 1H,1′H-2,2′-bibenzimidazole, derivatives of 2,2′-bi-1,3-benzoxazole, and derivatives of 2,2′-bi-1,3-benzothiazole.

39. An optical film according to claims 38, wherein the organic compound has general structural formulas selected from structures 16-34:

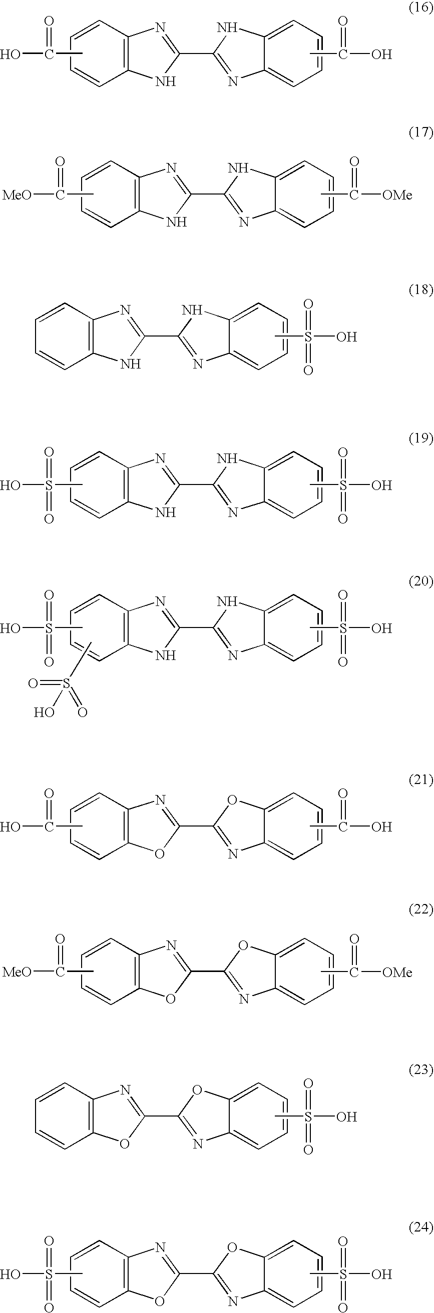

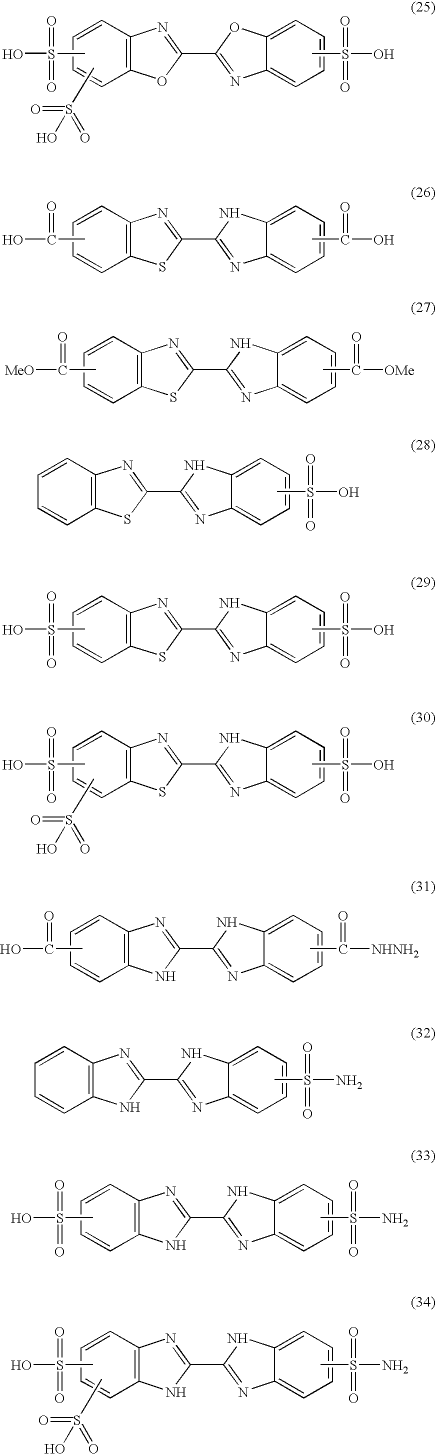

40. An optical film according to any of claims 34 to 39, wherein the longitudinal axes of the heterocyclic molecular systems have approximately isotropic alignment in the substrate plane.

41. An optical film according to any of claims 34 to 39, wherein the longitudinal axes of the heterocyclic molecular systems have approximately anisotropic alignment in the substrate plane.

42. An optical film according to any of claims 22 to 41, wherein said solid layer is generally a Uniaxial retardation layer possessing two refractive indices (nx and ny) corresponding to two mutually perpendicular directions in the plane of the substrate surface and one refractive index (nz) in the normal direction to the substrate surface, and wherein the refractive indices obey the following condition: nx=ny>nz.

43. An optical film according to any of claim 22 to 31 or 34 to 39 or 41, wherein said solid layer is generally a biaxial retardation layer possessing one refractive index (nz) in the normal direction to the substrate surface and two refractive indices (nx and ny) corresponding to two mutually perpendicular directions in the plane of the substrate surface and wherein the refractive indices obey the following condition: nx>ny>nz.

44. An optical film according to any of claims 22 to 43, wherein the substrate is made of a polymer.

45. An optical film according to any of claims 22 to 43, wherein the substrate is made of a glass.

46. An optical film according to any of claims 22 to 45, further comprising an antireflection or antiflashing coating on the rear surface of the substrate.

47. An optical film according to any of claims 22 to 46, wherein the substrate is transparent for electromagnetic radiation in the visible spectral range.

48. An optical film according to claim 47, wherein the transmission coefficient of the substrate in the visible spectral range is not less than 90%.

49. An optical film according to any of claims 22 to 48, further comprising a planarization transparent layer on the front surface of the substrate.

50. An optical film according to any of claims 22 to 45 or 47 to 49, further comprising a reflective layer on the rear surface of the substrate.

51. An optical film according to any of claim 22 to 45 or 49, wherein the substrate is a specular or diffusive reflector.

52. An optical film according to any of claim 22 to 45 or 49, wherein the substrate is a reflective polarizer.

53. An optical film according to any of claims 22 to 52, further comprising a substantially transparent adhesive layer applied on top of the solid layer.

54. An optical film according to claim 53, further comprising a protective coating applied on the transparent adhesive layer.

55. An optical film according to any of claims 22 to 54, comprising two or more solid layers, wherein these layers comprise different organic compounds of the general structural formula (III) ensuring a difference at least one of refraction indices (nx, ny, or nz) in two adjacent layers.

56. An optical film according to any of claims 22 to 55, wherein the solid layer is partially or entirely a crystal layer.

57. A method of producing an optical film, comprising the steps of
a) preparation of a solution of an organic compound of the general structural formula (I) or a salt thereof 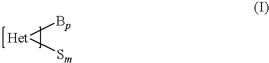
where Het is a predominantly planar heterocyclic molecular system possessing hydrophilic properties,
B is a binding group;
p is 3, 4, 5, 6, 7, or 8;
S is a molecular group providing solubility of the organic compound;
m is 0, 1, 2, 3, 4, 5, 6, 7, or 8;
wherein at least one fraction of said heterocyclic molecular system is capable of forming flat anisometric particles in the solution owing to lateral interaction of the binding groups via noncovalent chemical bonds;
b) application of a liquid layer of the solution onto a substrate,
wherein the liquid layer is substantially transparent for electromagnetic radiation in the visible spectral range from 400 to 700 nm and the flat anisometric particles and the heterocyclic molecular systems are bound among themselves owing to lateral interaction of the binding groups via non-covalent chemical bonds and are predominantly oriented in the plane of the substrate; and
c) drying to form a solid layer.

58. A method according to claim 57, wherein said predominantly planar heterocyclic molecular system is partially or completely conjugated.

59. A method according to any of claim 57 or 58, further comprising at least one additional substituent selected from a list comprising —CH₃, —C₂H₅, —NO₂, —Cl, —Br, —F, —CF₃, —CN, —NCS, —OH, —OCH₃, —OC₂H₅, —OCOCH₃, —OCN, —SCN, —NH₂, —NHCOCH₃, and —CONH₂.

60. A method according to any of claims 57 to 59, wherein the binding groups are selected from the list comprising hydrogen acceptor (A), hydrogen donor (D), and group having the general structural formula (II) 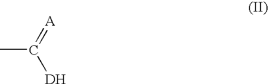
wherein the hydrogen acceptor (A) and hydrogen donor (D) are independently selected from the list comprising NH-group, and oxygen (O).

61. A method according to any of claims 57 to 60, wherein at least one of the binding groups is selected from the list comprising the hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is an alkyl group or an aryl group, the alkyl group having the general formula CₙH₂ₙ₊₁— where n is 1 to 23, preferably 1, 2, 3 or 4, the aryl group being selected from the group consisting of phenyl, benzyl and naphthyl.

62. A method according to claim 61, wherein the hetero-atoms are selected from the list comprising nitrogen, oxygen, sulfur, and any combination thereof.

63. A method according to any of claims 57 to 62, wherein at least one of the binding groups is a complementary group.

64. A method according to any of claims 57 to 63, wherein the heterocyclic molecular system has an axis of symmetry of order k (Cₖ) directed perpendicularly with respect to the plane of heterocyclic molecular system, where k is an integer number of no less than 3.

65. A method according to claim 64, wherein the predominantly planar heterocyclic molecular system comprises pyrazine or/and imidazole fragments and has a general structural formula selected from the list comprising structures 1-4:

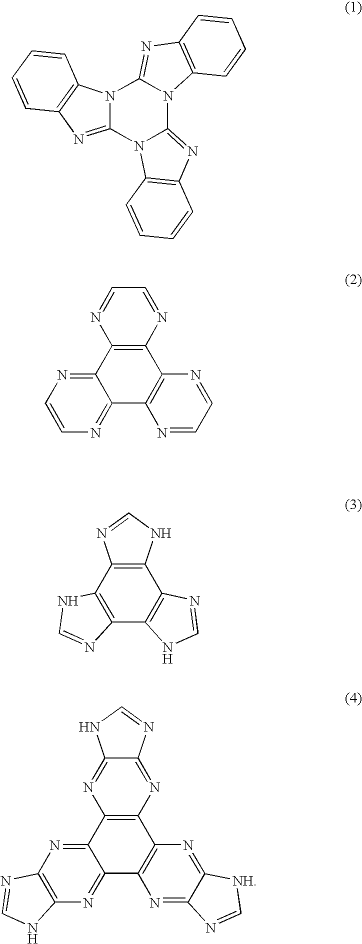

66. A method according to any of claims 57 to 63, wherein the heterocyclic molecular system has an extended anisometric form having a longitudinal axis.

67. A method according to claim 66, wherein the heterocyclic molecular system has a general structural formula selected from the list comprising structures 5-6, where E and G moieties are selected independently from the list comprising O, S, and NR⁴, where R⁴ is selected from the list comprising H, NH₂, OH:

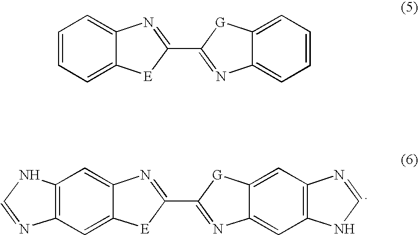

68. A method according to any of the claims 57 to 63, wherein the heterocyclic molecular system is an oligomer comprising imidazole or/and benzimidazole cycles, the imidazole or/and benzimidazole cycles being capable of forming hydrogen bonds.

69. A method according to claim 68, wherein the heterocyclic molecular system has a general structural formula selected from the list comprising structures 7-15, where n is the number in the range from 1 to 5:

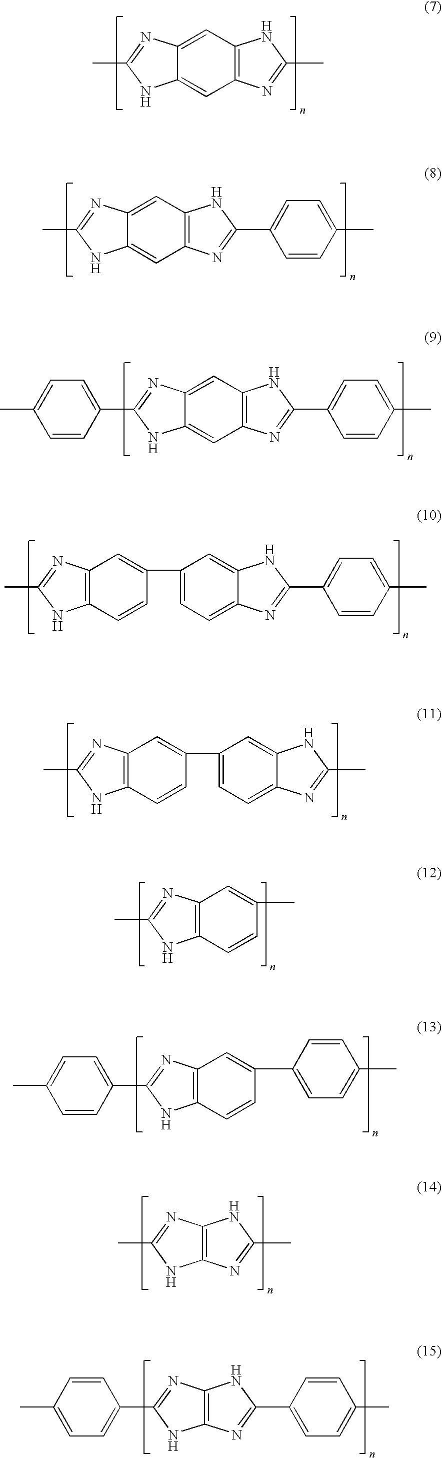

70. A method according to any of claims 57 to 63, wherein the organic compound is selected from the list comprising derivatives of 1H,1′H-2,2′-bibenzimidazole, derivatives of 2,2′-bi-1,3-benzoxazole, and derivatives of 2,2′-bi-1,3-benzothiazole.

71. A method according to claims 70, wherein the organic compound has general structural formulas selected from structures 16-34:

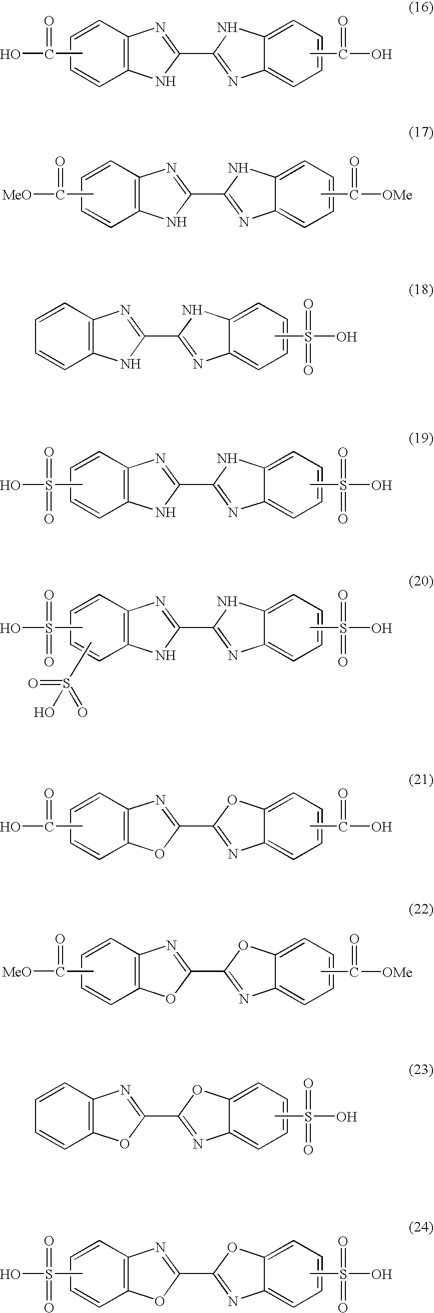

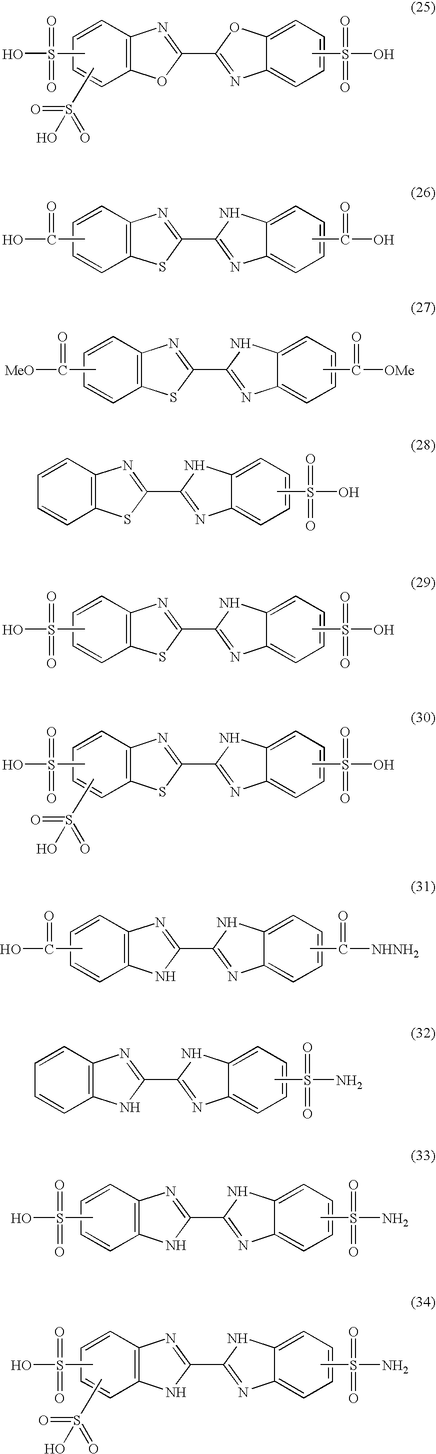

72. A method according to any of the claims 57 to 71, wherein the non-covalent chemical bond is selected from the list comprising a single hydrogen bond, dipole-dipole interaction, cation—pi-interaction, Van-der-Waals interaction, coordination bond, ionic bond, ion-dipole interaction, multiple hydrogen bond, interaction via the hetero-atoms and any combination thereof.

73. A method according to any of claims 57 to 72, wherein said liquid layer further comprises a solvent selected from the list comprising water, water-miscible solvent, and any combination thereof.

74. A method according to 73, wherein the water-miscible solvent is selected from the list comprising dimethylsulfoxide, dimethylformamide, acetone, acetylacetone, liquid amine, amine aqueous solution, alcohol, and any combination thereof.

75. A method according to any of claims 57 to 74, wherein the amount of solvent is controlled so as to provide the liquid-layer viscosity necessary for applying a liquid layer by means of a hydrodynamical flow.

76. A method according to claim 75, wherein the liquid-layer viscosity does not exceed 2 Pas.

77. A method according to any of claims 57 to 76, wherein the drying step is executed in airflow.

78. A method according to any of claims 57 to 77, further comprising a pretreatment step before the application onto the substrate.

79. A method according to claim 78, wherein the pretreatment comprises the step of making the surface of the substrate hydrophilic.

80. A method according to any of claim 78 or 79, wherein the pretreatment further comprises application of a planarization layer.

81. A method according to any of claims 57 to 80, further comprising a post-treatment step with a solution of any aqueous-soluble inorganic salt with a cation selected from the list comprising H⁺, Ba²⁺, Ca²⁺, Mg²⁺, Sr²⁺, La³⁺, Zn²⁺, Zr⁴⁺, Ce³⁺, Y³⁺, Yb³⁺, Gd³⁺ and any combination thereof.

82. A method according to claim 81, wherein the application of the liquid layer to the substrate step and the post-treatment step are carried out simultaneously.

83. A method according to any of claim 81 or 82, wherein the drying and, post-treatment steps are carried out simultaneously.

84. A method according to any of claim 81 or 82 wherein the post-treatment step is carried out after drying.

85. A method according to any of claims 57 to 84, wherein the solution is an isotropic solution.

86. A method according to any of claims 57 to 84, wherein the solution is a lyotropic liquid crystal solution.

87. A method according to any of claims 57 to 84, wherein the application is made using a gel.

88. A method according to any of claims 57 to 84, wherein the application is made using a viscous liquid phase.

89. A method according to any of claims 57 to 88, wherein the application and drying steps are repeated in a cyclical manner, and wherein the step of applying an alignment action onto said liquid layer on the substrate is performed after or simultaneously with the step of applying the liquid layer to the substrate, the organic compound in the liquid layer being either the same or different from that used in the previous cycle and having an absorption of electromagnetic radiation in at least one independently selected wavelength subrange of the UV spectral range.

90. A method according to any of claims 57 to 89, wherein the application and drying steps are repeated in a cyclical manner, and wherein the step of applying an alignment action onto said liquid layer on the substrate is performed after or simultaneously with the step of applying the liquid layer to the substrate, the organic compound in the liquid layer being different from that used in the previous cycle, at least one of the refractive indices (nx, ny, or nz) being different in two adjacent layers.

91. A method for the synthesis of 2,2′-bibenzheteroazole derivatives represented by the general structural formula (IV): 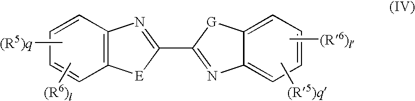
where q+l=0, 1, 2, 3 or 4; q′+l′=0, 1, 2, 3 or 4; E and G moieties are selected independently from the list comprising O, S, NR⁴, where R⁴ is selected from the list comprising H, NH₂, and OH, R⁵, R′⁵, R⁶ and R′⁶ are substituents selected independently from the list comprising —COOH, —COMe, —CO₂Me, —CONH₂, —CONHNH₂, —SO₃H, —SO₂NH₂, —SO₂—NH—SO₂—NH₂, which method comprises following steps:
a) reacting a component of formula (V) with a component selected from the list comprising structures (VII) and (VIII);
b) reacting the compound obtained in the previous step with a component of formula (VI); 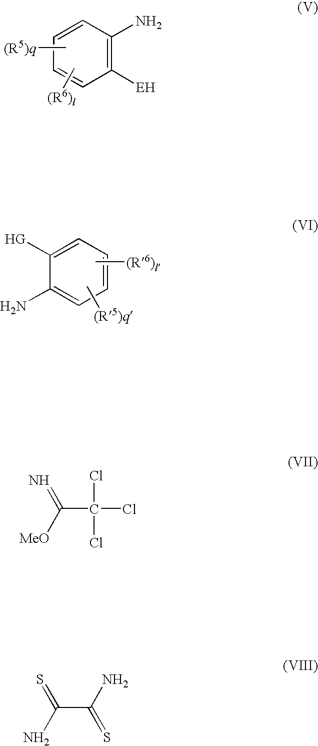
wherein the solvent is selected from the list comprising AcOH, DMF, MeOH, EtOH and mixtures thereof.

92. A method for the synthesis of 2,2′-bibenzheteroazole derivatives represented by the general structural formula (IV′): 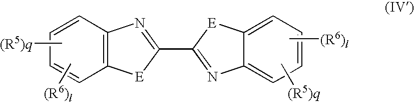
where q+l=0, 1, 2, 3 or 4; E moiety is selected from the list comprising O, S, NR⁴, where R⁴ is selected from the list comprising H, NH₂, OH, R⁵ and R⁶ are substituents selected independently from the list comprising —COOH, —COMe, —CO₂Me, —CONH₂, —CONHNH₂, —SO₃H, —SO₂NH₂, —SO₂NH—SO₂—NH₂, which method comprises following steps
a) reacting a component of formula (V) with a component selected from the list comprising structures (VII) and (VIII), and 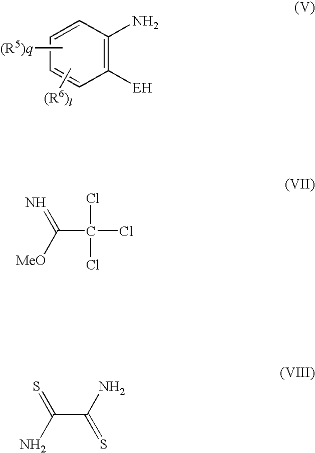
(b) stirring the mixture;
wherein the solvent is selected from the list comprising AcOH, DMF, MeOH, EtOH and mixtures thereof.

93. A method according to claim 92, further comprising the step of treating the mixture with Et₃N followed by HCl, wherein the treating step is simultaneous with, or subsequent to, the stirring step.

94. A 2,2′-bibenzheteroazole derivative of the general structural formula (IV) 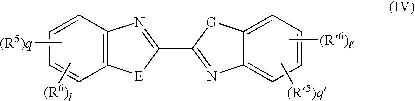
where q+l=0, 1, 2, 3 or 4; q′+l′=0, 1, 2, 3 or 4; E and G moieties are selected independently from the list comprising O, S, NR⁴, where R⁴ is selected from the list comprising H, NH₂, OH, R⁵, R′⁵, R⁶ and R′⁶ are substituents selected independently from the list comprising —COOH, —COMe, —CO₂Me, —CONH₂, —CONHNH₂, —SO₃H, —SO₂NH₂, and —SO₂—NH—SO₂—NH₂.

95. A 2,2′-bibenzheteroazole derivative according to claim 94 having a general structural formula from the list comprising structures 16 to 34:

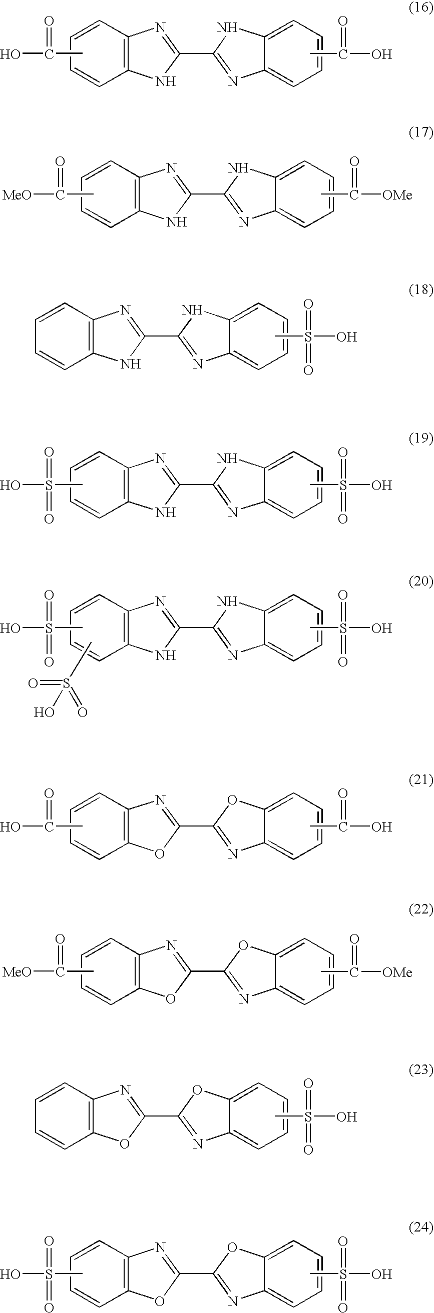

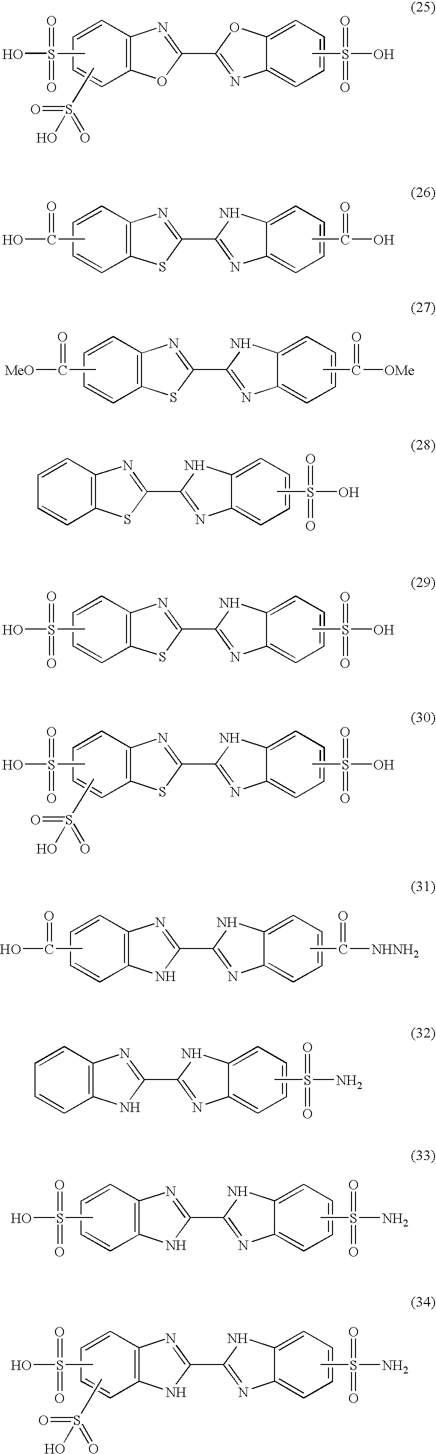

96. A method for the synthesis of tricarboxy-5,11,17-trimethyl-11,17-dihydro-5H-bisbenzimidazo[1′,2′:3,4; 1″,2″:5,6][1,3,5]triazino[1,2-a]benzimidazole-6,12,18-triium bromide represented by the general structural formula (IX): 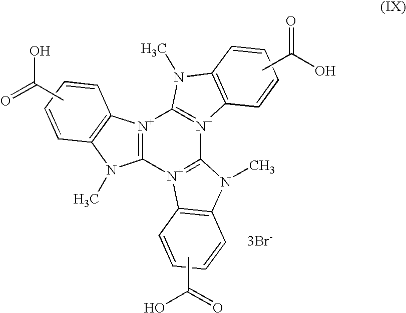
comprising the steps of:
a) bromination and methylation of 1H-benzimidazole-6-carboxylic acid to obtain 2-bromo-1-methyl-1H-benzimidazole-5(6)-carboxylic acids, and
b) condensation of the 2-bromo-1-methyl-1H-benzimidazole-5(6)-carboxylic acids obtained in step (a).

97. A method for the synthesis of a bisbenzimidazo[1′,2′:3,4;1″,2″:5,6][1,3,5]triazino[1,2-a]benzimidazole-tricarboxylic acid represented by the general structural formula (X): 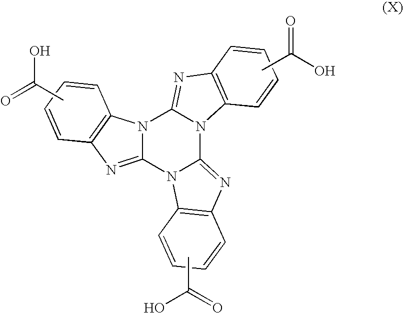
comprising the steps of:
a) preparation of methyl 3,4-diaminobenzoate dihydrochloride comprising the step of bubbling hydrogen chloride through a solution of 3,4-diaminobenzoic acid in methanol;
b) preparation of methyl 2-oxo-2,3-dihydro-1H-benzimidazole-5-carboxylate by condensation of methyl 3,4-diaminobenzoate dihydrochloride from step (a) with urea;
c) transformation of methyl 2-oxo-2,3-dihydro-1H-benzimidazole-5-carboxylate from step (b) to methyl 2-chloro-1H-benzimidazole-5(6)-carboxylate by treatment with hydrogen chloride and phosphorus oxychloride;
d) preparation of trimethyl bisbenzimidazo[1′,2′:3,4;1″,2″:5,6][1,3,5]triazino[1,2-a]benzimidazole-tricarboxylates by trimerization of obtained methyl 2-chloro-1H-benzimidazole-5(6)-carboxylate from step (c); and
e) alkaline hydrolysis of methyl esters of trimethyl bisbenzimidazo[1′,2′:3,4;1″,2″:5,6][1,3,5]triazino[1,2-a]benzimidazole-tricarboxylates from step (d) to obtain product (X).
